SEQUENCE LISTING (39 sequences)

SEQ ID NO: 1 - DNA, 22 bp, Triticum aestivum
  source 1..22: mol_type = genomic DNA
gatggtgacgtacgtgccctac

SEQ ID NO: 2 - DNA, 156 bp, Triticum aestivum
  source 1..156: mol_type = genomic DNA
ctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagc

SEQ ID NO: 3 - DNA, 152 bp, Triticum aestivum
  source 1..152: mol_type = other DNA
ctgcgcctgtacccggcggtgccgcaggaccccaagggcatcgcggaggacgacgtgctcccggacggcaccaaggtgcgcgccggcgggatggtgacgtgccctactccatggggcggatggagtacaactggggccccgacgccgccagc

SEQ ID NO: 4 - DNA, 157 bp, Triticum aestivum
  source 1..157: mol_type = other DNA
ctccgcctgtacccggcggtgccgcaggaccccaagggcatcgcggaggacgacgtgctcccggacggcaccaaggtgcgcgccggcgggatggtgacgtaccgtgccctactccatggggcggatggagtacaactggggccccgacgccgccagc

SEQ ID NO: 5 - DNA, 147 bp, Triticum aestivum
  source 1..147: mol_type = other DNA
ctccgcctgtacccggcggtgccgcaggaccccaagggcatcgcggaggacgacgtgctcccggacggcaccaaggtgcgcgccggcgggatggtgacgtactccatggggcggatggagtacaactggggccccgacgccgccagc

SEQ ID NO: 6 - DNA, 75 bp, Triticum aestivum
  source 1..75: mol_type = other DNA
ctccgcctgtacccggcggtgccgcaggaccccaagggcatcgcggaggacgacgtgctcccggacggcaccaag

SEQ ID NO: 7 - DNA, 133 bp, Triticum aestivum
  source 1..133: mol_type = other DNA
ctccgcctgtacccggcggtgccgcaggaccccaagggcatcgcggaggacgacgtgctcccggacggcaccaaggtacgtgccctactccatggggcggatggagtacaactggggccccgacgccgccagc

SEQ ID NO: 8 - DNA, 66 bp, Triticum aestivum
  source 1..66: mol_type = other DNA
ctgcgcctgtacgtgccctactccatggggcggatggagtacaactggggccccgacgccgccagc

SEQ ID NO: 9 - DNA, 60 bp, Triticum aestivum
  source 1..60: mol_type = other DNA
ctgcgcctgtacccggcggtgccgcaggaccccaagggcatcgcggaggacgacgtcggc

SEQ ID NO: 10 - protein, 9 aa, simian virus 40
MAPKKKRKV

SEQ ID NO: 11 - protein, 18 aa, Agrobacterium tumefaciens
KRPRDRHDGELGGRKRAR

SEQ ID NO: 12 - DNA, 21 bp, synthetic construct
  misc_feature 1..21: note = Synthetic Construct
  source 1..21: mol_type = other DNA
gacgtacgtgccctactccat

SEQ ID NO: 13 - DNA, 20 bp, synthetic construct
  misc_feature 1..20: note = Synthetic Construct
  source 1..20: mol_type = other DNA
acgtacgtgccctactccat

SEQ ID NO: 14 - DNA, 19 bp, synthetic construct
  misc_feature 1..19: note = Synthetic Construct
  source 1..19: mol_type = other DNA
acgtacgtgccctactcca

SEQ ID NO: 15 - DNA, 20 bp, synthetic construct
  misc_feature 1..20: note = Synthetic Construct
  source 1..20: mol_type = other DNA
atcgagctcggggaggccgg

SEQ ID NO: 16 - DNA, 16 bp, synthetic construct
  misc_feature 1..16: note = Synthetic Construct
  source 1..16: mol_type = other DNA
atgaagagccccatgg

SEQ ID NO: 17 - DNA, 21 bp, synthetic construct
  misc_feature 1..21: note = Synthetic Construct
  source 1..21: mol_type = other DNA
gacgtacgtgccctactccat

SEQ ID NO: 18 - DNA, 20 bp, synthetic construct
  misc_feature 1..20: note = Synthetic Construct
  source 1..20: mol_type = other DNA
gacgtacgtgccctactcca

SEQ ID NO: 19 - DNA, 56 bp, Oryza sativa
  source 1..56: mol_type = genomic DNA
ccggcgggatggtgacgtacgtgccctactccatggggaggatggagtacaactgg

SEQ ID NO: 20 - DNA, 55 bp, Oryza sativa
  source 1..55: mol_type = other DNA
ccggcgggatggtgacgtacgtgccctactcatggggaggatggagtacaactgg

SEQ ID NO: 21 - DNA, 55 bp, Oryza sativa; identical to SEQ ID NO: 20
  source 1..55: mol_type = other DNA

SEQ ID NO: 22 - DNA, 55 bp, Oryza sativa; identical to SEQ ID NO: 20
  source 1..55: mol_type = other DNA

SEQ ID NO: 23 - DNA, 54 bp, Oryza sativa
  source 1..54: mol_type = other DNA
ccggcgggatggtgacgtacgtgccctactatggggaggatggagtacaactgg

SEQ ID NO: 24 - DNA, 44 bp, Oryza sativa
  source 1..44: mol_type = other DNA
ccggcgggatggtgacgtacatggggaggatggagtacaactgg

SEQ ID NO: 25 - DNA, 49 bp, Oryza sativa
  source 1..49: mol_type = other DNA
ccggcgggatggtgacgtacgtgccctggggaggatggagtacaactgg

SEQ ID NO: 26 - DNA, 44 bp, Oryza sativa; identical to SEQ ID NO: 24
  source 1..44: mol_type = other DNA

SEQ ID NO: 27 - DNA, 44 bp, Oryza sativa; identical to SEQ ID NO: 24
  source 1..44: mol_type = other DNA

SEQ ID NO: 28 - DNA, 55 bp, Oryza sativa; identical to SEQ ID NO: 20
  source 1..55: mol_type = other DNA

SEQ ID NO: 29 - DNA, 53 bp, Oryza sativa
  source 1..53: mol_type = other DNA
ccggcgggatggtgacgtacgtgccctacttggggaggatggagtacaactgg

SEQ ID NO: 30 - DNA, 53 bp, Oryza sativa; identical to SEQ ID NO: 29
  source 1..53: mol_type = genomic DNA

SEQ ID NO: 31 - DNA, 1934 bp, Triticum urartu
  exon 1..336
  exon 425..640
  exon 725..1021
  exon 1115..1912
  source 1..1934: mol_type = genomic DNA
atggaggaagctcacggcggcatgccgtcgacgacgacggcgttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtcaccgtcgacatgcccttcacctcctacacctacatcgccgaccccgtgaacgtcgagcatgtgctcaagaccaatttcaacaattaccccaaggtgaaacaatcctcgagatgtcagacaaggttcagtaatcggtactgacagtgttacaaatgtctgaaatctgaaattgtatgtctagggggaggtgtacaggtcctacatggacgtgctgctcggcgacggcatattcaacgccgacggcgagctctggaggaagcagaggaagacggcgagcttcgagttcgcttccaagaacctgagagacttcagcacgatcgtgttcagggagtactcgctgaagctgtccagcatcctgagccaggcttgcaaggccggcaaagtcgtggacatgcaggcaactgaactcattcccttggtcatctgaacgttgatttcttggacaaaatttcaagattctgacgcgagcggacgaattcaggagctgtacatgaggatgacgctggactcgatctgcaaggtcgggttcggggtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaaggtgcgtgcgtgatcatcgtcattcgtcaagctccggatcgctggtttgtgtagtaggtgccattgatcactgacacgttaactgggtgcgcagatcaagcacgacatactgtcgcggttcatcgagctgggcgaggccggcggcgacgacggcggcagcctgttcggggacgacaagggcctccgcgacgtggtgctcaacttcgtgatcgccgggcgggacaccacggccacgacgctgtcctggttcacctacatggccatgacgcacccggccgtggccgagaagctccgccgcgagctggccgccttcgaggcggatcgcgcccgcgaggagggcgtcgctctggtcccctgcagcgacggcgagggcgccgacgaggccttcgccgcccgcgtggcgcagttcgcggggctcctgagctacgacgggctcgggaagctggtgtacctccacgcgtgcgtgacggagacgctgcggctgtacccggcggtgccgcaggaccccaagggcatcgcggaggacgacgtgctcccggacggcaccaaggtgcgcgccggcgggatggtgacgtacgtgccctactccatggggcggatggagtataactggggccccgacgccgccagcttccggccggagcggtggatcggcgacgacggcgcgttccgcaacgcgtcgccgttcaagttcacggcgttccaggcggggccgcggatctgcctcggcaaggactcggcgtacctgcagatgaagatggcgctggccatactgtgcaggttcttcaggttcgagctcgtggagggccaccccgtcaagtaccgcatgatgaccatcctctccatggcgcacggcctcaaggtccgcgtctccagggcgccgctcgcctgatcttgatctgggttccggcgag

SEQ ID NO: 32 - DNA, 1929 bp, Aegilops speltoides
  exon 1..333
  exon 424..639
  exon 724..1020
  exon 1111..1908
  source 1..1929: mol_type = genomic DNA
atggaggaagctcaccttggcatgccgtcgacgacggccttcttcccgctggcagggctccacaagttcatggccatcttcctcgtgttcctctcgtggatcctggtccactggtggagcctgaggaagcagaaggggccgaggtcatggccggtcatcggcgccacgctggagcagctgaggaactactaccggatgcacgactggctcgtggagtacctgtccaagcaccggacggtcaccgtcgacatgcccttcacctcctacacctacatcgccgacccggtgaacgtcgagcatgtgctcaagaccaacttcaacaattaccccaaggtgaaacaatcctcgagatgtcagtcaaggttcggtataatcggtactgacagtgttacaaatgtctgaaatctgaaattgtgtgtgtagggggaggtgtacaggtcctacatggacgtgctgctcggcgacggcatattcaacgccgacggcgagctctggaggaagcagaggaagacggcgagcttcgagttcgcttccaagaacctgagagacttcagcacgatcgtgttccgggagtactccctgaagctgtccagcatcctgagccaggcttgcaaggccggcaaagttgtggacatgcaggtaactgaactctttcccttggtcatctgaacgttgatttcttggacaaaatttcaagattgtgacgcgagcgagccaattcaggagctgtacatgaggatgacgctggactcgatctgcaaggtggggttcggggtggagatcggcacgctgtcgccggagctgccggagaacagcttcgcgcaggccttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaaggtgcgtacgcggtcatcgtcattcgtcaagctcccgatcgctggtttgtgcagatgccattgatcactgacacattaactgggcgcgcagatcaagcacgacatactgtcgcggttcatcgagctgggcgaggccggcggcgacgacggcggcagcctgttcggggacgacaagggcctccgcgacgtggtgctcaacttcgtgatcgccgggcgggacaccacggccacgacgctctcctggttcacctacatggccatgacgcacccggacgtggccgagaagctccgccgcgagctggccgccttcgagtccgagcgcgcccgcgaggagggcgtcgctctggtcccctgcagcgacggcgagggctccgacgaggccttcgccgcccgcgtggcgcagttcgcggggctcctgagctacgacgggctcgggaagctggtgtacctccacgcgtgcgtgacggagacgctgcgcctgtacccggcggtgccgcaggatcccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgatggcgccttccgcaacgcgtcgccgttcaagttcacggcgttccaggcggggccgcggatctgcctgggcaaggactcggcgtacctgcagatgaagatggcgctggccatcctgtgcaggttcttcaggttcgagctcgtggagggccaccccgtcaagtaccgcatgatgaccatcctctccatggcgcacggcctcaaggtccgcgtctccagggcgccgctcgcctgatcttgatctggttccggcgag

SEQ ID NO: 33 - DNA, 1930 bp, Aegilops squarrosa
  exon 1..333
  exon 424..639
  exon 724..1020
  exon 1111..1908
  source 1..1930: mol_type = genomic DNA
atggaggaagctcacggcggcatgccgtcgacgacggccttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtgaccgtcgacatgcccttcacctcctacacctacatcgccgacccggtgaacgtcgagcatgtgctcaagaccaacttcaacaattaccccaaggtgaaacaatcctcgagatgtcagtaaaggttcagtataatcggtactgacagtgttacaaatgtctgaaatctgaaattgtatgtgtagggggaggtgtacaggtcctacatggacgtgctgctcggcgacggcatattcaacgccgacggcgagctctggaggaagcagaggaagacggcgagcttcgagttcgcttccaagaacttgagagacttcagcacgatcgtgttcagggagtactccctgaagctgtccagcatactgagccaggcttgcaaggccggcaaagttgtggacatgcaggtaactgaactcattcccttggtcatctgaacgttgatttcttggacaaaatttcaagattctgacgcgagcgagcgaattcaggagctgtatatgaggatgacgctggactcgatctgcaaagtggggttcggagtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaaggtgcgtgcgtggtcatcgtcattcgtcaagctcccggtcgctggtttgtgtagatgccatggatcactgacacactaactgggcgcgcagatcaagcacgacatactgtcgcggttcatcgagctgggcgaggccggcggcgacgacggcggcagtctgttcggggacgacaagggcctccgcgacgtggtgctcaacttcgtgatcgccgggcgggacaccacggccacgacgctgtcctggttcacctacatggccatgacgcacccggacgtggccgagaagctccgccgcgagctggccgccttcgaggcggagcgcgcccgcgaggatggcgtcgctctggtcccctgcggcgacggcgagggctccgacgaggccttcgctgcccgcgtggcgcagttcgcggggttcctgagctacgacggcctcgggaagctggtgtacctccacgcgtgcgtgacggagacgctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgacggcgccttccgcaacgcgtcgccgttcaagttcacggcgttccaggcggggccgcggatttgcctcggcaaggactcggcgtacctgcagatgaagatggcgctggcaatcctgtgcaggttcttcaggttcgagctcgtggagggccaccccgtcaagtaccgcatgatgaccatcctctccatggcgcacggcctcaaggtccgcgtctccagggcgccgctcgcctgatcttgatctggttccggcgagg

SEQ ID NO: 34 - DNA, 1581 bp, Triticum urartu
  exon 1..384
  exon 462..746
  exon 821..988
  exon 1080..1484
  source 1..1581: mol_type = genomic DNA
atggaagagaagaagccgcggcggcagggagccgcaggacgcgatggcatcgtgcagtacccgcacctcttcatcgcggccctggcgctggccctggtcctcatggaccccttccacctcggcccgctggccgggatcgactaccggccggtgaagcacgagctggcgccgtacagggaggtcatgcagcgctggccgagggacaacggcagccgcctcaggctcggcaggctcgagttcgtcaacgaggtgttcgggccggagtccatcgagttcgaccgccagggccgcgggccctacgccgggctcgccgacggccgcgtcgtgcggtggatgggggacaaggccgggtgggagacgttcgccgtcatgaatcctgactggtattggcttactgcagaaaaaccatagcttacctgtgtgtgtgcaaactaaaatagtttctttcggaaaaaaaaaggtcggagaaagtttgtgctaacggagtggagtcgacgacgaagaagcagcacgggaaggagaagtggtgcggccggcctctcgggctgaggttccacagggagaccggcgagctcttcatcgccgacgcgtactatgggctcatggccgttggcgaaagcggcggcgtggcgacctccctggcgagggaggccggcggggacccggtccacttcgccaacgacctcgacatccacatgaacggctcgatattcttcaccgacacgagcacgagatacagcagaaagtgagcggagtactgctgccgatctcctttttctgttcttgagatttgtgtttgacaaatgactgatcatgcagggaccatttgaacattttgctggaaggagaaggcacggggaggctgctgagatatgaccgagaaaccggtgccgttcatgtcgtgctcaacgggctggtcttcccaaacggcgtgcagatctcacaggaccagcaatttctcctcttctccgagacaacaaactgcaggtgagataaactcaggttttcagtatgatccggctcgagagatccaggaactgatgacgcctttattaatcggctcatgcatgcacactaggatcatgaggtactggctggaaggtccaagagcgggccaggtggaggtgttcgcgaacctgccggggttccccgacaacgtgcgcttgaacagcaaggggcagttctgggtggcgatcgactgctgccggacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtggtcgaggtactcgaggaccggggcggcgaggtgatgaagctggtgagcgaggtgagggaggtggaccggaggctgtggatcgggaccgttgcgcacaaccacatcgccacgatcccttacccgttggactagagtgtgtagtgtctcatttgatttgctggttttatattagcaaggaggtgtatcagtttatggtttgcttgtttattgggttcgtgtgatgatcgtg

SEQ ID NO: 35 - DNA, 1536 bp, Aegilops speltoides
  exon 1..384
  exon 461..745
  exon 841..996
  exon 1075..1479
  source 1..1536: mol_type = genomic DNA
atggaagagaagaagccgcggcggcagggagccgcagtacgcgatggcatcgtgcagtacccgcacctcttcatcgcggccctggcgctggccctggtcgtcatggaccccttccacctcggcccgctggccgggatcgactaccggccggtgaagcacgagctggcgccgtacagggaggtcatgcagcgctggccgagggacaacggcagccggctgagactcggcaggctcgagttcgtcaacgaggtgttcgggccggagtccatcgagttcgaccgccagggccgcgggccctacgccggcctcgccgacggccgcgtcgtgcggtggatgggggagaaggccgggtgggagacgttcgccgtcatgaatcctgactggtattggcttactgcagataaatccatagcttacctgtgtgtttgcaaactaaaatggtttcttggaaaaaaaaaggtcggagaaagtttgtgctaacggagtggagtcaacgacgaagaagcagcacgggaaggagaagtggtgcggccggcctctcgggctgaggttccacagggagaccggcgagctcttcatcgccgacgcgtactatgggctcatggccgtcggcgaaagcggcggcgtggcgacctccctggcaagggaggccggcggggacccggtccacttcgccaacgaccttgacatccacatgaacggctcgatattcttcaccgacacgagcacgagatacagcagaaagtgagcgaactgctgccgctgttctccatttttgttaatgagatgttgtgtttgagtgtctgacaccatgactgatcatgcagggaccatttgaacattttgctggaaggagaaggcacggggaggctgctgagatatgaccgagaaaccggtgccgttcatgtcgtgctcaacgggctggtcttcccaaacggcgtgcagatttcacaggaccagcaatttctcctcttctccgagacaacaaactgcaggtgagataaactcagattttcagtatgatccggctcgagagatccaggaactgatgacggctcatgcacgcacgctaggatcatgaggtactggctggaaggtccaagagcgggccaggtggaggtgttcgcgaacctgccggggttccccgacaacgtgcgcctgaacagcaaggggcagttctgggtggcgatcgactgctgccggacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtcgtggaggtgctcgaggaccggggcggcgaggtgatgaagctggtgagcgaggtgagggaggtggaccggaggctgtggatcgggaccgttgcgcacaaccacatcgccacgatcccttacccgctggactagagggagtgtgtagtgtccatttgctggtttatattagcaaggaggtgtatcagttta

SEQ ID NO: 36 - DNA, 1573 bp, Aegilops squarrosa
  exon 1..384
  exon 463..747
  exon 822..989
  exon 1068..1472
  source 1..1573: mol_type = genomic DNA
atggaagagaagaaaccgcggcggcagggagccgcagtacgcgatggcatcgtgcagtacccgcacctcttcatcgcggccctggcgctggccctggtcctcatggacccgttccacctcggcccgctggccgggatcgactaccgaccggtgaagcacgagctggcgccgtacagggaggtcatgcagcgctggccgagggacaacggcagccgcctcaggctcggcaggctcgagttcgtcaacgaggtgttcgggccggagtccatcgagttcgaccgccagggccgcgggccttacgccgggctcgccgacggccgcgtcgtgcggtggatgggggacaaggccgggtgggagacgttcgccgtcatgaatcctgactggtactggcttactgcagaaaaacccatagcttacctgtgtgtgtgcagactaaaatagtttctttcataaaaaaaaaggtcggagaaagtttgtgctaacggagtggagtcgacgacgaagaagcagcacgggaaggagaagtggtgcggccggcctctcggcctgaggttccacagggagaccggcgagctcttcatcgccgacgcgtactatgggctcatggccgtcggcgaaaggggcggcgtggcgacctccctggcgagggaggccggcggggacccggtccacttcgccaacgaccttgacatccacatgaacggctcgatattcttcaccgacacgagcacgagatacagcagaaagtgagcggagtactgctgccgatctcctttttctgttcttgagatttgtgtttgacaaatgactgatcatgcagggaccatttgaacattttgctggaaggagaaggcacggggaggctgctgagatatgaccgagaaaccggtgccgttcatgtcgtgctcaacgggctggtcttcccaaacggcgtgcagatatcacaggaccagcaatttctcctcttctccgagacaacaaactgcaggtgagataaactcaggttttcagtatgatccggctcgagagatccaggaactgatgacggctcatgcatgcacactaggatcatgaggtactggctggaaggtccaagagcgggccaggtggaggtgttcgcgaacctgccggggttccccgacaatgtgcgcctgaacagcaaggggcagttctgggtggccatcgactgctgccgtacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtcgtggaggtgctcgaggaccggggcggcgaggtgatgaagctggtgagcgaggtgagggaggtggaccggaggctgtggatcgggaccgttgcgcacaaccacatcgccacgatcccttacccgctggactagagggagtgtgtagtgtcccatttgatttgctggttttatattagcaaggaggtgtatcagtttatggtttgcttgttcattgggttcgtgtgatgatcgtg

SEQ ID NO: 37 - DNA, 414 bp, Triticum urartu
  exon 1..414
  source 1..414: mol_type = genomic DNA
atgttgaggatgcagcagcaggtggagggcgtggtgggcggcggcatcgtggccgaggcggaggaggcggcggtgtacgagcgggtggctcgcatggccagcggcaacgcggtggtcgtcttcagcgccmgcggctgctgcatgtgccacgtcgtcaagcgcctcctgcttggcctgggggtcggccccaccgtctacgagttggaccagatgggcggcgccgggcgagagatccaggcggcgctggcgcagctgctgccccccggacccggcgccggccaccaccagcagccgccagtgcccgtggtgttcgtcggcgggaggctcctgggcggcgtggagaaggtcatggcgtgccacatcaacggcacgctcgtcccgctcctcaaggacgccggcgcgctctggctctga

SEQ ID NO: 38 - DNA, 414 bp, Aegilops speltoides
  exon 1..414
  source 1..414: mol_type = genomic DNA
atgttgaggatgcagcagcaggtggagggcgtggtgggcggcggcatcgtggcggaggcggaggaggcggccgtgtacgagcgggtggctcgcatggccagcggcaacgcggtggtcgtcttcagcgccagcggctgctgcatgtgccacgtcgtcaagcgcctcctgcttggcctgggagtcggccccaccgtgtacgagttggaccagatgggcggcgccgggcgggagatccaggcggccctggcgcagctgctgccccccggacccggcgccggccaccaccagcagccgccggtgcccgtggtgttcgtyggcgggaggctcctgggcggcgtggagaaggtgatggcgtgccacatcaacggcacgctcgtcccgctcctcaaggacgccggcgcgctctggctctga

SEQ ID NO: 39 - DNA, 414 bp, Aegilops squarrosa
  exon 1..414
  source 1..414: mol_type = genomic DNA
atgttgaggatgcagcagcaggtggagggcgtggtgggcggcggcatcatggcggaggcggaggaggcggcggtgtacgagcgggtggctcgcatggccagcggcaacgcggtggtcgtcttcagcgccagcggctgctgcatgtgccacgtcgtcaagcgcctcctgcttggcctgggggtcggccccaccgtctacgagttggaccagatgggcggcgccgggcgagagatccaggcggcgctggcgcagctgctgccccccggacccggcgccggccaccaccagcagccgccagtgcccgtggtgttcgtcggcgggaggctcctgggcggcgtggagaaggtgatggcgtgccacatcaacggcacgctcgtcccgctcctcaaggacgccggcgcgctctggctctga

(19) United States
(12) Patent Application Publication
(10) Pub. No.: US 2025/0207148 A1
(43) Pub. Date: Jun. 26, 2025

(54) RESTORATION OF MALE FERTILITY IN WHEAT

(71) Applicant: PIONEER HI-BRED INTERNATIONAL, INC, JOHNSTON, IA (US)

(72) Inventors: ANDREW MARK CIGAN, MADISON, WI (US); MANJIT SINGH, JOHNSTON, IA (US)

(73) Assignee: PIONEER HI-BRED INTERNATIONAL, INC, JOHNSTON, IA (US)

(21) Appl. No.: 19/027,066

(22) Filed: Jan. 17, 2025

Related U.S. Application Data
(63) Continuation of application No. 15/536,336, filed on Jun. 15, 2017.
(60) Provisional application No. 62/092,604, filed on Dec. 16, 2014.

(51) Int. Cl. C12N 15/82; C07K 14/415; C12N 9/02; C12N 15/01

(52) U.S. Cl. CPC C12N 15/8289; C07K 14/415; C12N 9/0077; C12N 15/01; C12N 15/8213

(57) ABSTRACT

Manipulation of male fertility in a polyploid species requires attention to the interaction of male-fertility alleles of multiple genomes. In hexaploid wheat, single-genome heterozygotes for Ms26 provide differential levels of male fertility across genomes. Hexaploid wheat homozygous for mutations in the Ms26 gene on the A, B, and D genomes is male-sterile. Male fertility may be restored by sufficient levels of expression of Ms26 using native Ms26 or a transgene, which may be native to wheat or to another species, or a combination of native and transgenic alleles. CRISPR/Cas9 technology may be used to generate mutations in Ms26 in wheat or rice.

### FIELD OF THE INVENTION

The present invention relates to the field of plant molecular biology, more particularly to influencing male fertility.

### REFERENCE TO ELECTRONICALLY-SUBMITTED SEQUENCE LISTING

The official copy of the sequence listing is submitted electronically as an ASCII formatted sequence listing file named 6596WOPCT_ST25.txt, created on Dec. 15, 2015, having a size of 59 KB, and is filed concurrently with the specification. The sequence listing contained in this ASCII formatted document is part of the specification and is herein incorporated by reference in its entirety.

### BACKGROUND OF THE INVENTION

Development of hybrid plant breeding has made possible considerable advances in quality and quantity of crops produced. Increased yield and combination of desirable characteristics, such as resistance to disease and insects, heat and drought tolerance, along with variations in plant composition are all possible because of hybridization procedures. These procedures frequently rely heavily on providing for a male parent contributing pollen to a female parent to produce the resulting hybrid.

Field crops are bred through techniques that take advantage of the plant's method of pollination. A plant is considered self-pollinated if pollen from one flower is transferred to the same or another flower of the same plant or a genetically identical plant. A plant is considered cross-pollinated if the pollen comes from a flower on a genetically different plant.

In certain species, such as Brassica campestris, the plant is normally self-sterile and can only be cross-pollinated. In predominantly self-pollinating species, such as soybeans, wheat, and cotton, the male and female reproductive organs are anatomically juxtaposed such that during natural pollination, the male reproductive organs of a given flower pollinate the female reproductive organs of the same flower.

Bread wheat (Triticum aestivum) is a hexaploid plant having three pairs of homologous chromosomes defining genomes A, B and D. The endosperm of wheat grain comprises two haploid complements from a maternal reproductive cell and one from a paternal reproductive cell. The embryo of wheat grain comprises one haploid complement from each of the maternal and paternal reproductive cells. Hexaploidy has been considered a significant obstacle in researching and developing useful variants of wheat. In fact, very little is known regarding how homeologous genes of wheat interact, how their expression is regulated, and how the different proteins produced by homeologous genes function separately or in concert. Strategies for manipulation of expression of male-fertility polynucleotides in wheat will require consideration of the ploidy level of the individual wheat variety. Triticum aestivum is a hexaploid containing three genomes designated A, B, and D (N=21); each genome comprises seven pairs of nonhomologous chromosomes. Einkorn wheat varieties are diploids (N=7) and emmer wheat varieties are tetraploids (N=14).

### BRIEF SUMMARY OF THE INVENTION

Compositions and methods for modulating male fertility in wheat are provided. Compositions comprise expression cassettes comprising one or more male-fertility polynucleotides, or fragments or variants thereof, operably linked to a promoter, wherein expression of the polynucleotide modulates the male fertility of a plant. Various methods are provided wherein the level and/or activity of a polynucleotide or polypeptide that influences male fertility is modulated in a plant or plant part. Compositions and methods provide approaches to complement and restore male fertility to wheat plants containing mutations in genes important to sporophytic production of pollen and enabling the production of hybrid wheat plants.

### DESCRIPTION OF THE FIGURES

FIG. 1 shows an alignment of the NHEJ mutations induced by the MS26+ homing endonuclease. The top sequence is the MS26 target site (SEQ ID NO: 1) compared to a reference sequence (SEQ ID NO: 2) which illustrates the unmodified locus. Deletions as a result of imperfect NHEJ are shown by a “-”, while the gap represented in the MS26 target site (SEQ ID NO: 1), the reference MS26 sequence (SEQ ID NO: 2) and SEQ ID NOs 3, 5-9 corresponds to a single C nucleotide insertion present in SEQ ID NO: 4. The mutations were identified by sequencing of subcloned PCR products in DNA vectors.

FIG. 2 shows flowers and anthers of wild-type, triple homozygous ms26 mutant, and single heterozygous (Ms26/ms26) double homozygous mutant (ms26/ms26) wheat plants. A: Flowers from wild-type (left) and triple homozygous ms26 mutant (right). Cross section of wild-type (B) and triple homozygous ms26 (C) anthers staged at late vacuolate microspore development. D-F: Cross section of anthers staged at late vacuolate microspore development from single genome heterozygous (Ms26/ms26), double homozygous (ms26/ms26); G-I: close-up of cross sections displayed in D-F, respectively.

### DETAILED DESCRIPTION

The present disclosure now will be described more fully hereinafter; some, but not all embodiments are shown. Indeed, the disclosure may be embodied in many different forms and should not be construed as limited to the embodiments set forth herein; rather, these embodiments are provided so that this disclosure will satisfy applicable legal requirements.

Many modifications and other embodiments of the disclosure will come to mind to one skilled in the art, having the benefit of the teachings presented in the descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### I. Male-Fertility Polynucleotides

Sexually reproducing plants develop specialized tissues for the production of male and female gametes. Successful production of male gametes relies on proper formation of the male reproductive tissues. The stamen, which embodies the male reproductive organ of plants, contains various parts and cell types, including for example, the filament, anther, tapetum, and pollen. As used herein, “male tissue” refers to the specialized tissue in a sexually reproducing plant that is responsible for production of the male gamete. Male tissues include, but are not limited to, the stamen, filament, anther, tapetum, microspores and pollen.

The process of mature pollen grain formation begins with microsporogenesis, wherein meiocytes are formed in the sporogenous tissue of the anther. Microgametogenesis follows, wherein microspores divide mitotically and develop into the microgametophyte, or pollen grains. The condition of “male fertility” or “male fertile” refers to those plants producing a mature pollen grain capable of fertilizing a female gamete to produce a subsequent generation of offspring. The term “influences male fertility” or “modulates male fertility”, as used herein, refers to any increase or decrease in the ability of a plant to produce a mature pollen grain when compared to an appropriate control. A “mature pollen grain” or “mature pollen” refers to any pollen grain capable of fertilizing a female gamete to produce a subsequent generation of offspring. Likewise, the term “male-fertility polynucleotide” or “male-fertility polypeptide” refers to a polynucleotide or polypeptide that modulates male fertility. A male-fertility polynucleotide may, for example, encode a polypeptide that participates in the process of microsporogenesis or microgametogenesis.

Certain alleles of male sterility genes such as MAC1, EMS1 or GNE2 (Sorensen et al. (2002) Plant J. 29:581-594) prevent cell growth in the quartet stage. Mutations in the SPOROCYTELESS/NOZZLE gene act early in development, but impact both anther and ovule formation such that plants are male and female sterile. The SPOROCYTELESS gene of Arabidopsis is required for initiation of sporogenesis and encodes a novel nuclear protein (Genes Dev. 1999 Aug. 15;13 (16): 2108-17).

Isolated or substantially purified nucleic acid molecules or protein compositions are disclosed herein. An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the polypeptides disclosed herein or biologically active portion thereof is recombinantly produced, optimally culture medium represents less than about 30%, 20%, 10%, 5%, or 1% (by dry weight) of chemical precursors or non-protein-of-interest chemicals.

A “subject plant” or “subject plant cell” is one in which genetic alteration, such as transformation, has been effected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or plant cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

Fragments and variants of the disclosed polynucleotides and proteins encoded thereby are also provided. By “fragment” is intended a portion of the polynucleotide or a portion of the amino acid sequence and hence protein encoded thereby. Fragments of a polynucleotide may encode protein fragments that retain the biological activity of the native protein and hence influence male fertility; these fragments may be referred to herein as “active fragments.” Alternatively, fragments of a polynucleotide that are useful as hybridization probes or which are useful in constructs and strategies for down-regulation or targeted sequence modification generally do not encode protein fragments retaining biological activity, but may still influence male fertility. Thus, fragments of a nucleotide sequence may range from at least about 20 nucleotides, about 50 nucleotides, about 100 nucleotides, up to the full-length polynucleotide encoding a polypeptide disclosed herein.

A fragment of a polynucleotide that encodes a biologically active portion of a polypeptide that influences male fertility will encode at least 15, 25, 30, 50, 100, 150, or 200 contiguous amino acids, or up to the total number of amino acids present in a full-length polypeptide that influences male fertility. Fragments of a male-fertility polynucleotide that are useful as hybridization probes or PCR primers, or in a down-regulation construct or targeted-modification method generally need not encode a biologically active portion of a polypeptide but may influence male fertility.

Thus, a fragment of a male-fertility polynucleotide as disclosed herein may encode a biologically active portion of a male-fertility polypeptide, or it may be a fragment that can be used as a hybridization probe or PCR primer or in a downregulation construct or targeted-modification method using methods known in the art or disclosed below. A biologically active portion of a male-fertility polypeptide can be prepared by isolating a portion of one of the male-fertility polynucleotides disclosed herein, expressing the encoded portion of the male-fertility protein (e.g., by recombinant expression in vitro), and assessing the activity of the encoded portion of the male-fertility polypeptide.

“Variants” is intended to mean substantially similar sequences. For polynucleotides, a variant comprises a deletion and/or addition of one or more nucleotides at one or more sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of a male-fertility polypeptide disclosed herein. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis, and which may encode a male-fertility polypeptide.

Variants of a particular polynucleotide disclosed herein (i.e., a reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein.

“Variant” protein is intended to mean a protein derived from the native protein by deletion or addition of one or more amino acids at one or more sites in the native protein and/or substitution of one or more amino acids at one or more sites in the native protein. Variant proteins disclosed herein are biologically active, that is they continue to possess biological activity of the native protein, that is, male fertility activity as described herein. Such variants may result from, for example, genetic polymorphism or human manipulation. A biologically active variant of a protein disclosed herein may differ from that protein by as few as 1-15 amino acid residues, as few as 1-10, such as 6-10, as few as 5, as few as 4, 3, 2, or even 1 amino acid residue.

The proteins disclosed herein may be altered in various ways including amino acid substitutions, deletions, truncations, and insertions. Methods for such manipulations are generally known in the art. For example, amino acid sequence variants and fragments of the male-fertility polypeptides can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides disclosed herein include both the naturally occurring sequences as well as DNA sequence variants. Likewise, the male-fertility polypeptides and proteins encompass both naturally-occurring polypeptides as well as variations and modified forms thereof. Such polynucleotide and polypeptide variants may continue to possess the desired male-fertility activity, in which case the mutations that will be made in the DNA encoding the variant must not place the sequence out of reading frame and optimally will not create complementary regions that could produce secondary mRNA structure. See, EP Patent Application Publication No. 75,444.

Variant functional polynucleotides and proteins also encompass sequences and proteins derived from a mutagenic and recombinogenic procedure such as DNA shuffling. With such a procedure, one or more different male fertility sequences can be manipulated to create a new male-fertility polypeptide possessing desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the male-fertility polynucleotides disclosed herein and other known male-fertility polynucleotides to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Variant nucleic acid sequences can be made by introducing sequence changes randomly along all or part of a genic region, including, but not limited to, chemical or irradiation mutagenesis and oligonucleotide-mediated mutagenesis (OMM) (Bectham et al. 1999; Okuzaki and Toriyama 2004). Alternatively or additionally, sequence changes can be introduced at specific selected sites using double-strand-break technologies such as but not limited to ZNFs, custom designed homing endonucleases, TALENs, CRISPR/CAS (also referred to as guide RNA/Cas endonuclease systems (U.S. patent application Ser. No. 14/463,687 filed on Aug. 20, 2014)), or other protein-, or polynucleotide-, or coupled polynucleotide-protein-based mutagenesis technologies. The resultant variants can be screened for altered gene activity. It will be appreciated that the techniques are often not mutually exclusive. Indeed, the various methods can be used singly or in combination, in parallel or in series, to create or access diverse sequence variants.

### II. Sequence Analysis

As used herein, “sequence identity” or “identity” in the context of two polynucleotide or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

The use of the term “polynucleotide” is not intended to limit the present disclosure to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides disclosed herein also encompass all forms of sequences including, but not limited to, single-stranded forms, double-stranded forms, hairpins, stem-and-loop structures, and the like.

III. Expression cassettes

A male-fertility polynucleotide disclosed herein can be provided in an expression cassette for expression in an organism of interest. The cassette can include 5′ and 3′ regulatory sequences operably linked to a male-fertility polynucleotide as disclosed herein. “Operably linked” is intended to mean a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame.

The expression cassettes disclosed herein may include in the 5′-3′ direction of transcription, a transcriptional and translational initiation region (i.e., a promoter), a polynucleotide of interest, and a transcriptional and translational termination region (i.e., termination region) functional in the host cell (e.g., a plant cell). Expression cassettes are also provided with a plurality of restriction sites and/or recombination sites for insertion of the male-fertility polynucleotide to be under the transcriptional regulation of the regulatory regions described elsewhere herein. The regulatory regions (i.e., promoters, transcriptional regulatory regions, and translational termination regions) and/or the polynucleotide of interest may be native/analogous to the host cell or to each other. Alternatively, the regulatory regions and/or the polynucleotide of interest may be heterologous to the host cell or to each other. As used herein, “heterologous” in reference to a polynucleotide or polypeptide sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, unless otherwise specified, a chimeric polynucleotide comprises a coding sequence operably linked to a transcription initiation region that is heterologous to the coding sequence.

In certain embodiments the polynucleotides disclosed herein can be stacked with any combination of polynucleotide sequences of interest or expression cassettes as disclosed elsewhere herein or known in the art. For example, the male-fertility polynucleotides disclosed herein may be stacked with any other polynucleotides encoding male-gamete-disruptive polynucleotides or polypeptides, cytotoxins, markers, or other male fertility sequences as disclosed elsewhere herein or known in the art. The stacked polynucleotides may be operably linked to the same promoter as the male-fertility polynucleotide, or may be operably linked to a separate promoter polynucleotide.

As described elsewhere herein, expression cassettes may comprise a promoter operably linked to a polynucleotide of interest, along with a corresponding termination region. The termination region may be native to the transcriptional initiation region, may be native to the operably linked male-fertility polynucleotide of interest or to the male-fertility promoter sequences, may be native to the plant host, or may be derived from another source (i.e., foreign or heterologous). Convenient termination regions are available from the Ti-plasmid of A. tumefaciens, such as the octopine synthase and nopaline synthase termination regions. See also Guerincau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides of interest may be optimized for increased expression in the transformed plant. That is, the polynucleotides can be synthesized or altered to use plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165 (2): 233-238), MDMV leader (Maize Dwarf Mosaic Virus) (Johnson et al. (1986) Virology 154:9-20), and human immunoglobulin heavy-chain binding protein (BiP) (Maccjak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

In particular embodiments, the expression cassettes disclosed herein comprise a promoter operably linked to a male-fertility polynucleotide, or fragment or variant thereof, as disclosed herein.

In certain embodiments, plant promoters can preferentially initiate transcription in certain tissues, such as stamen, anther, filament, and pollen, or developmental growth stages, such as sporogenous tissue, microspores, and microgametophyte. Such plant promoters are referred to as “tissue-preferred,” “cell-type-preferred,” or “growth-stage preferred.” Promoters which initiate transcription only in certain tissue are referred to as “tissue-specific.” Likewise, promoters which initiate transcription only at certain growth stages are referred to as “growth-stage-specific.” A “cell-type-specific” promoter drives expression only in certain cell types in one or more organs, for example, stamen cells, or individual cell types within the stamen such as anther, filament, or pollen cells.

A “male-fertility promoter” may initiate transcription exclusively or preferentially in a cell or tissue involved in the process of microsporogenesis or microgametogenesis. Male-fertility polynucleotides disclosed herein, and active fragments and variants thereof, can be operably linked to male-tissue-specific or male-tissue-preferred promoters including, for example, stamen-specific or stamen-preferred promoters, anther-specific or anther-preferred promoters, pollen-specific or pollen-preferred promoters, tapetum-specific promoters or tapetum-preferred promoters, and the like. Promoters can be selected based on the desired outcome. For example, the polynucleotides of interest can be operably linked to constitutive, tissue-preferred, growth stage-preferred, or other promoters for expression in plants.

In one embodiment, the promoters may be those which express an operably-linked polynucleotide of interest exclusively or preferentially in the male tissues of the plant. No particular male-fertility tissue-preferred or tissue-specific promoter must be used in the process; and any of the many such promoters known to one skilled in the art may be employed. One such promoter is the 5126 promoter, which preferentially directs expression of the polynucleotide to which it is linked to male tissue of the plants, as described in U.S. Pat. Nos. 5,837,851 and 5,689,051. Other examples include the maize Ms45 promoter described at U.S. Pat. No. 6,037,523; SF3 promoter described at U.S. Pat. No. 6,452,069; the BS92-7 promoter described at WO 02/063021; an SGB6 regulatory element described at U.S. Pat. No. 5,470,359; the TA29 promoter (Koltunow, et al., (1990) Plant Cell 2:1201-1224; Nature 347:737 (1990); Goldberg, et al., (1993) Plant Cell 5:1217-1229 and U.S. Pat. No. 6,399,856); an SB200 gene promoter (WO 2002/26789), a PG47 gene promoter (U.S. Pat. No. 5,412,085; U.S. Pat. No. 5,545,546; Plant J 3 (2): 261-271 (1993)), a G9 gene promoter (U.S. Pat. Nos. 5,837,850; 5,589,610); the type 2 metallothionein-like gene promoter (Charbonnel-Campaa, et al., Gene (2000) 254:199-208); the Brassica Bca9 promoter (Lee, et al., (2003) Plant Cell Rep. 22:268-273); the ZM13 promoter (Hamilton, et al., (1998) Plant Mol. Biol. 38:663-669); actin depolymerizing factor promoters (such as Zmabp1, Zmabp2; see, for example Lopez, et al., (1996) Proc. Natl. Acad. Sci. USA 93:7415-7420); the promoter of the maize pectin methylesterase-like gene, ZmC5 (Wakeley, et al., (1998) Plant Mol. Biol. 37:187-192); the profilin gene promoter Zmprol (Kovar, et al., (2000) The Plant Cell 12:583-598); the sulphated pentapeptide phytosulphokine gene ZmPSK1 (Lorbiecke, et al., (2005) Journal of Experimental Botany 56 (417): 1805-1819); the promoter of the calmodulin binding protein Mpcbp (Reddy, et al., (2000) J. Biol. Chem. 275 (45): 35457-70).

As disclosed herein, constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Patent Nos. 5,608, 149; 5,608,144; 5,604, 121; 5,569,597; 5,466,785; 5,399,680; 5,268,463; 5,608,142; and 6,177,611.

“Seed-preferred” promoters include both those promoters active during seed development, such as promoters of seed storage proteins, as well as those promoters active during seed germination. See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase) (sec WO 00/11177 and U.S. Pat. No. 6,225,529; herein incorporated by reference). Gamma-zein is an endosperm-specific promoter. Globulin-1 (Glob-1) is a representative embryo-specific promoter. For dicots, seed-specific promoters include, but are not limited to, bean β-phascolin, napin, B-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, globulin 1, etc. See also WO 00/12733, where seed-preferred promoters from end1 and end2 genes are disclosed. Additional embryo specific promoters are disclosed in Sato et al. (1996) Proc. Natl. Acad. Sci. 93:8117-8122; Nakase et al. (1997) Plant J 12:235-46; and Postma-Haarsma et al. (1999) Plant Mol. Biol. 39:257-71. Additional endosperm specific promoters are disclosed in Albani et al. (1984) EMBO 3:1405-15; Albani et al. (1999) Theor. Appl. Gen. 98:1253-62; Albani et al. (1993) Plant J. 4:343-55; Mena et al. (1998) The Plant Journal 116:53-62, and Wu et al. (1998) Plant Cell Physiology 39:885-889.

Dividing cell or meristematic tissue-preferred promoters have been disclosed in Ito et al. (1994) Plant Mol. Biol. 24:863-878; Reyad et al. (1995) Mo. Gen. Genet. 248:703-711; Shaul et al. (1996) Proc. Natl. Acad. Sci. 93:4868-4872; Ito et al. (1997) Plant J. 11:983-992; and Trehin et al. (1997) Plant Mol. Biol. 35:667-672.

Stress inducible promoters include salt/water stress-inducible promoters such as P5CS (Zang et al. (1997) Plant Sciences 129:81-89); cold-inducible promoters, such as, cor15a (Hajela et al. (1990) Plant Physiol. 93:1246-1252), cor15b (Wlihelm et al. (1993) Plant Mol Biol 23:1073-1077), wsc120 (Ouellet et al. (1998) FEBS Lett. 423-324-328), ci7 (Kirch et al. (1997) Plant Mol Biol. 33:897-909), ci21A (Schneider et al. (1997) Plant Physiol. 113:335-45); drought-inducible promoters, such as, Trg-31 (Chaudhary et al (1996) Plant Mol. Biol. 30:1247-57), rd29 (Kasuga et al. (1999) Nature Biotechnology 18:287-291); osmotic inducible promoters, such as, Rab17 (Vilardell et al. (1991) Plant Mol. Biol. 17:985-93) and osmotin (Raghothama et al. (1993) Plant Mol Biol 23:1117-28); and, heat inducible promoters, such as, heat shock proteins (Barros et al. (1992) Plant Mol. 19:665-75; Marrs et al. (1993) Dev. Genet. 14:27-41), and smHSP (Waters et al. (1996) J. Experimental Botany 47:325-338). Other stress-inducible promoters include rip2 (U.S. Pat. No. 5,332,808 and U.S. Publication No. 2003/0217393) and rd29A (Yamaguchi-Shinozaki et al. (1993) Mol. Gen. Genetics 236:331-340).

As discussed elsewhere herein, the expression cassettes comprising male-fertility polynucleotides may be stacked with other polynucleotides of interest. Any polynucleotide of interest may be stacked with the male-fertility polynucleotide.

Male-fertility polynucleotides disclosed herein may be stacked in or with expression cassettes comprising a promoter operably linked to a polynucleotide which is male-gamete-disruptive; that is, a polynucleotide which interferes with the function, formation, or dispersal of male gametes. A male-gamete-disruptive polynucleotide can operate to prevent function, formation, or dispersal of male gametes by any of a variety of methods. By way of example but not limitation, this can include use of polynucleotides which encode a gene product such as DAM-methylase or barnase (See, for example, U.S. Pat. No. 5,792,853 or 5,689,049; PCT/EP89/00495); encode a gene product which interferes with the accumulation of starch, degrades starch, or affects osmotic balance in pollen, such as alpha-amylase (See, for example, US. Pat. Nos. 7,875,764; 8,013,218; 7,696,405, 8,614,367); inhibit formation of a gene product important to male gamete function, formation, or dispersal (See, for example, U.S. Pat. Nos. 5,859,341; 6,297,426); encode a gene product which combines with another gene product to prevent male gamete formation or function (See, for example, U.S. Pat. Nos. 6,162,964; 6,013,859; 6,281,348; 6,399,856; 6,248,935; 6,750,868; 5,792,853); are antisense to, or cause co-suppression of, a gene critical to male gamete function, formation, or dispersal (See, for example, U.S. Pat. Nos. 6, 184,439; 5,728,926; 6, 191,343; 5,728,558; 5,741,684); interfere with expression of a male-fertility polynucleotide through use of hairpin formations (See, for example, Smith et al. (2000) Nature 407:319-320; WO 99/53050 and WO 98/53083) or the like.

Male-gamete-disruptive polynucleotides include dominant negative genes such as methylase genes and growth-inhibiting genes. Sec, U.S. Pat. No. 6,399,856. Dominant negative genes include diphtheria toxin A-chain gene (Czako and An (1991) Plant Physiol. 95 687-692; Greenfield et al. (1983) PNAS 80:6853); cell cycle division mutants such as CDC in maize (Colasanti et al. (1991) PNAS 88:3377-3381); the WT gene (Farmer et al. (1994) Mol. Genet. 3:723-728); and P68 (Chen et al. (1991) PNAS 88:315-319).

Further examples of male-gamete-disruptive polynucleotides include, but are not limited to, pectate lyase gene pelE from Erwinia chrysanthermi (Kenn et al (1986) J. Bacteriol. 168:595); CytA toxin gene from Bacillus thuringiensis Israeliensis (McLean et al (1987) J. Bacteriol. 169:1017 (1987), U.S. Pat. No. 4,918,006); DNAses, RNAses, proteases, or polynucleotides expressing anti-sense RNA. A male-gamete-disruptive polynucleotide may encode a protein involved in inhibiting pollen-stigma interactions, pollen tube growth, fertilization, or a combination thereof.

Male-fertility polynucleotides disclosed herein may be stacked with expression cassettes disclosed herein comprising a promoter operably linked to a polynucleotide of interest encoding a reporter or marker product. Examples of suitable reporter polynucleotides known in the art can be found in, for example, Jefferson et al. (1991) in Plant Molecular Biology Manual, ed. Gelvin et al. (Kluwer Academic Publishers), pp. 1-33; DeWet et al. Mol. Cell. Biol. 7:725-737 (1987); Goff et al. EMBO J. 9:2517-2522 (1990); Kain et al. BioTechniques 19:650-655 (1995); and Chiu et al. Current Biology 6:325-330 (1996). In certain embodiments, the polynucleotide of interest encodes a selectable reporter. These can include polynucleotides that confer antibiotic resistance or resistance to herbicides. Examples of suitable selectable marker polynucleotides include, but are not limited to, genes encoding resistance to chloramphenicol, methotrexate, hygromycin, streptomycin, spectinomycin, bleomycin, sulfonamide, bromoxynil, glyphosate, and phosphinothricin.

In some embodiments, the expression cassettes disclosed herein comprise a polynucleotide of interest encoding scorable or screenable markers, where presence of the polynucleotide produces a measurable product. Examples include a B-glucuronidase, or uidA gene (GUS), which encodes an enzyme for which various chromogenic substrates are known (for example, U.S. Pat. Nos. 5,268,463 and 5,599,670); chloramphenicol acetyl transferase, and alkaline phosphatase. Other screenable markers include the anthocyanin/flavonoid polynucleotides including, for example, a R-locus polynucleotide, which encodes a product that regulates the production of anthocyanin pigments (red color) in plant tissues, the genes which control biosynthesis of flavonoid pigments, such as the maize C1 and C2, the B gene, the p1 gene, and the bronze locus genes, among others. Further examples of suitable markers encoded by polynucleotides of interest include the cyan fluorescent protein (CYP) gene, the yellow fluorescent protein gene, a lux gene, which encodes a luciferase, the presence of which may be detected using, for example, X-ray film, scintillation counting, fluorescent spectrophotometry, low-light video cameras, photon counting cameras or multiwell luminometry, a green fluorescent protein (GFP), and DsRed2 (Clontech Laboratories, Inc., Mountain View, California), where plant cells transformed with the marker gene fluoresce red in color, and thus are visually selectable. Additional examples include a p-lactamase gene encoding an enzyme for which various chromogenic substrates are known (e.g., PADAC, a chromogenic cephalosporin), a xylE gene encoding a catechol dioxygenase that can convert chromogenic catechols, and a tyrosinase gene encoding an enzyme capable of oxidizing tyrosine to DOPA and dopaquinone, which in turn condenses to form the easily detectable compound melanin.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschmidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not meant to be limiting. Any selectable marker gene can be used in the compositions and methods disclosed herein.

In some embodiments, the expression cassettes disclosed herein comprise a first polynucleotide of interest encoding a male-fertility polynucleotide operably linked to a first promoter polynucleotide, stacked with a second polynucleotide of interest encoding a male-gamete-disruptive gene product operably linked to a male-tissue-preferred promoter polynucleotide. In certain embodiments, the expression cassettes described herein may also be stacked with a third polynucleotide of interest encoding a marker polynucleotide operably linked to a promoter polynucleotide.

In specific embodiments, the expression cassettes disclosed herein comprise a first polynucleotide of interest encoding a male fertility gene operably linked to a constitutive promoter, such as the cauliflower mosaic virus (CaMV) 35S promoter. The expression cassettes may further comprise a second polynucleotide of interest encoding a male-gamete-disruptive gene product operably linked to a male-tissue-preferred promoter. In certain embodiments, the expression cassettes disclosed herein may further comprise a third polynucleotide of interest encoding a marker gene, such as a herbicide resistance gene, operably linked to a constitutive promoter, such as the cauliflower mosaic virus (CaMV) 35S promoter.

### IV. Plants

### A. Plants Having Altered Levels/Activity of Male-Fertility Polypeptide

Further provided are plants having altered levels and/or activities of a male-fertility polypeptide and/or altered levels of male fertility. In some embodiments, the plants disclosed herein have stably incorporated into their genomes a heterologous male-fertility polynucleotide, or an active fragment or variant thereof, as disclosed herein.

Plants are further provided comprising the expression cassettes disclosed herein comprising a male-fertility polynucleotide operably linked to a promoter that is active in the plant. In some embodiments, expression of the male-fertility polynucleotide modulates male fertility of the plant. In certain embodiments, expression of the male-fertility polynucleotide increases male fertility of the plant. In certain embodiments, expression cassettes comprising a heterologous male-fertility polynucleotide as disclosed herein, or an active fragment or variant thereof, operably linked to a promoter active in a plant, are provided to a male-sterile plant. Upon expression of the heterologous male-fertility polynucleotide, male fertility is conferred; this may be referred to as restoring the male fertility of the plant. In specific embodiments, the plants disclosed herein comprise an expression cassette comprising a heterologous male-fertility polynucleotide as disclosed herein, or an active fragment or variant thereof, operably linked to a promoter, stacked with one or more expression cassettes comprising a polynucleotide of interest operably linked to a promoter active in the plant. For example, the stacked polynucleotide of interest can comprise a male-gamete-disruptive polynucleotide and/or a marker polynucleotide.

Plants disclosed herein may also comprise stacked expression cassettes described herein comprising at least two polynucleotides such that the at least two polynucleotides are inherited together in more than 50% of meioses, i.e., not randomly. Accordingly, when a plant or plant cell comprising stacked expression cassettes with two polynucleotides undergoes meiosis, the two polynucleotides segregate into the same progeny (daughter) cell. In this manner, stacked polynucleotides will likely be expressed together in any cell for which they are present. For example, a plant may comprise an expression cassette comprising a male-fertility polynucleotide stacked with an expression cassette comprising a male-gamete-disruptive polynucleotide such that the male-fertility polynucleotide and the male-gamete-disruptive polynucleotide are inherited together. Specifically, a male sterile plant could comprise an expression cassette comprising a male-fertility polynucleotide disclosed herein operably linked to a constitutive promoter, stacked with an expression cassette comprising a male-gamete-disruptive polynucleotide operably linked to a male-tissue-preferred promoter, such that the plant produces mature pollen grains. However, in such a plant, development of pollen comprising the male-fertility polynucleotide will be inhibited by expression of the male-gamete-disruptive polynucleotide.

### B. Plants and Methods of Introduction

As used herein, the term plant includes plant cells, plant protoplasts, plant cell tissue cultures from which a plant can be regenerated, plant calli, plant clumps, and plant cells that are intact in plants or parts of plants such as embryos, pollen, ovules, seeds, leaves, flowers, branches, fruit, kernels, cars, cobs, husks, stalks, roots, root tips, anthers, grain and the like. As used herein, by “grain” is intended the mature seed produced by commercial growers for purposes other than growing or reproducing the species. Progeny, variants, and mutants of the regenerated plants are also included within the scope of the disclosure, provided that these parts comprise the introduced nucleic acid sequences.

The methods disclosed herein comprise introducing a polypeptide or polynucleotide into a plant cell. “Introducing” is intended to mean presenting to the plant the polynucleotide or polypeptide in such a manner that the sequence gains access to the interior of a cell. The methods disclosed herein do not depend on a particular method for introducing a sequence into the host cell, only that the polynucleotide or polypeptides gains access to the interior of at least one cell of the host. Methods for introducing polynucleotide or polypeptides into host cells (i.e., plants) are known in the art and include, but are not limited to, stable transformation methods, transient transformation methods, and virus-mediated methods.

“Stable transformation” is intended to mean that the nucleotide construct introduced into a host (i.e., a plant) integrates into the genome of the plant and is capable of being inherited by the progeny thereof. “Transient transformation” is intended to mean that a polynucleotide or polypeptide is introduced into the host (i.e., a plant) and expressed temporally.

Transformation protocols as well as protocols for introducing polypeptides or polynucleotide sequences into plants may vary depending on the type of plant or plant cell, e.g., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cercals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kappler et al. (1990) Plant Cell Reports 9:415-418 and Kappler et al. (1992) Theor. Appl. Genet. 84:560-566 (whisker-mediated transformation); D′Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the male-fertility polynucleotides or expression cassettes disclosed herein can be provided to a plant using a variety of transient transformation methods. Such transient transformation methods include, but are not limited to, the introduction of the male-fertility polypeptide or variants and fragments thereof directly into the plant or the introduction of a male fertility transcript into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91:2176-2180 and Hush et al. (1994) The Journal of Cell Science 107:775-784, all of which are herein incorporated by reference. Alternatively, the male-fertility polynucleotide or expression cassettes disclosed herein can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use of particles coated with polyethylimine (PEI; Sigma #P3143).

In other embodiments, the male-fertility polynucleotides or expression cassettes disclosed herein may be introduced into plants by contacting plants with a virus or viral nucleic acids. Generally, such methods involve incorporating a nucleotide construct disclosed herein within a viral DNA or RNA molecule. It is recognized that a male fertility sequence disclosed herein may be initially synthesized as part of a viral polyprotein, which later may be processed by proteolysis in vivo or in vitro to produce the desired recombinant protein. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, a polynucleotide disclosed herein can be contained in a transfer cassette flanked by two non-identical recombination sites. The transfer cassette is introduced into a plant having stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be pollinated with either the same transformed strain or a different strain, and the resulting progeny having desired expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a male-fertility polynucleotide disclosed herein, for example, an expression cassette disclosed herein, stably incorporated into their genome.

The terms “target site”, “target sequence”, “target DNA”, “target locus”, “genomic target site”, “genomic target sequence”, and “genomic target locus” are used interchangeably herein and refer to a polynucleotide sequence in the genome (including chloroplast and mitochondrial DNA) of a cell at which a double-strand break is induced in the cell genome. The target site can be an endogenous site in the genome of a cell or organism, or alternatively, the target site can be heterologous to the cell or organism and thereby not be naturally occurring in the genome, or the target site can be found in a heterologous genomic location compared to where it occurs in nature. As used herein, terms “endogenous target sequence” and “native target sequence” are used interchangeably herein to refer to a target sequence that is endogenous or native to the genome of a cell or organism and is at the endogenous or native position of that target sequence in the genome of a cell or organism. Cells include plant cells as well as plants and seeds produced by the methods described herein.

In one embodiments, the target site, in association with the particular gene editing system that is being used, can be similar to a DNA recognition site or target site that is specifically recognized and/or bound by a double-strand-break-inducing agent, such as but not limited to a Zinc Finger endonuclease, a meganuclease, a TALEN endonuclease, a CRISPR-Cas guideRNA or other polynucleotide guided double strand break reagent.

The terms “artificial target site” and “artificial target sequence” are used interchangeably herein and refer to a target sequence that has been introduced into the genome of a cell or organism. Such an artificial target sequence can be identical in sequence to an endogenous or native target sequence in the genome of a cell but be located in a different position (i.e., a non-endogenous or non-native position) in the genome of a cell or organism.

The terms “altered target site”, “altered target sequence”, “modified target site”, and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Certain embodiments comprise polynucleotides disclosed herein which are modified using endonucleases. Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex.

Endonucleases also include meganucleases, also known as homing endonucleases (HEases). Like restriction endonucleases, HEases bind and cut at a specific recognition site. However, the recognition sites for meganucleases are typically longer, about 18 bp or more. (See patent publication WO2012/129373 filed on Mar. 22, 2012). Meganucleases have been classified into four families based on conserved sequence motifs (Belfort M, and Perlman P S J. Biol. Chem. 1995;270:30237-30240). These motifs participate in the coordination of metal ions and hydrolysis of phosphodiester bonds. HEases are notable for their long recognition sites, and for tolerating some sequence polymorphisms in their DNA substrates.

The naming convention for meganucleases is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr. Op. Biotechnol. 5:521-7; and Sadowski (1993) FASEB 7:760-7. In some examples the recombinase is from the Integrase or Resolvase families.

TAL effector nucleases are a class of sequence-specific nucleases that can be used to make double-strand breaks at specific target sequences in the genome of a plant or other organism. (Miller et al. (2011) Nature Biotechnology 29:143-148). Zinc finger nucleases (ZFNs) are engineered double-strand-break-inducing agents comprised of a zinc finger DNA binding domain and a double-strand-break-inducing agent domain. Recognition site specificity is conferred by the zinc finger domain, which typically comprises two, three, or four zinc fingers, for example having a C2H2 structure; however other zinc finger structures are known and have been engineered. Zinc finger domains are amenable for designing polypeptides which specifically bind a selected polynucleotide recognition sequence. ZFNs include engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognizes a sequence of 9 contiguous nucleotides; with a dimerization requirement of the nuclease, two sets of zinc finger triplets are used to bind an 18-nucleotide recognition sequence.

CRISPR loci (Clustered Regularly Interspaced Short Palindromic Repeats) (also known as SPIDRs—SPacer Interspersed Direct Repeats) constitute a family of recently described DNA loci. CRISPR loci consist of short and highly conserved DNA repeats (typically 24 to 40 bp, repeated from 1 to 140 times-also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

CRISPR loci were first recognized in E. coli (Ishino et al. (1987) J. Bacterial. 169:5429-5433; Nakata et al. (1989) J. Bacterial. 171:3553-3556). Similar interspersed short sequence repeats have been identified in Haloferax mediterranei, Streptococcus pyogenes, Anabaena, and Mycobacterium tuberculosis (Groenen et al. (1993) Mol. Microbiol. 10:1057-1065; Hoe et al. (1999) Emerg. Infect. Dis. 5:254-263; Masepohl et al. (1996) Biochim. Biophys. Acta 1307:26-30; Mojica et al. (1995) Mol. Microbiol. 17:85-93). The CRISPR loci differ from other SSRs by the structure of the repeats, which have been termed short regularly spaced repeats (SRSRs) (Janssen et al. (2002) OMICS J. Integ. Biol. 6:23-33; Mojica et al. (2000) Mol. Microbiol. 36:244-246). The repeats are short elements that occur in clusters, that are always regularly spaced by variable sequences of constant length (Mojica et al. (2000) Mol. Microbiol. 36:244-246).

Cas gene relates to a gene that is generally coupled, associated or close to or in the vicinity of flanking CRISPR loci. The terms “Cas gene”, “CRISPR-associated (Cas) gene” are used interchangeably herein. A comprehensive review of the Cas protein family is presented in Haft et al. (2005) Computational Biology, PLOS Comput Biol 1 (6): c60. doi: 10.1371/journal.pcbi.0010060. As described therein, 41 CRISPR-associated (Cas) gene families are described, in addition to the four previously known gene families. It shows that CRISPR systems belong to different classes, with different repeat patterns, sets of genes, and species ranges. The number of Cas genes at a given CRISPR locus can vary between species.

Cas endonuclease relates to a Cas protein encoded by a Cas gene, wherein said Cas protein is capable of introducing a double strand break into a DNA target sequence. The Cas endonuclease is guided by a guide polynucleotide to recognize and optionally introduce a double strand break at a specific target site into the genome of a cell (U.S. Provisional Application No. 62/023,239, filed Jul. 11, 2014). The guide polynucleotide/Cas endonuclease system includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA if a correct protospacer-adjacent motif (PAM) is approximately oriented at the 3′ end of the target sequence.

The Cas endonuclease gene can be Cas9 endonuclease, or a functional fragment thereof, such as but not limited to, Cas9 genes listed in SEQ ID NOs: 462, 474, 489, 494, 499, 505, and 518 of WO2007/025097 published Mar. 1, 2007. The Cas endonuclease gene can be a plant, maize or soybean optimized Cas9 endonuclease, such as but not limited to a plant codon optimized streptococcus pyogenes Cas9 gene that can recognize any genomic sequence of the form N (12-30) NGG. The Cas endonuclease can be introduced directly into a cell by any method known in the art, for example, but not limited to transient introduction methods, transfection and/or topical application.

As used herein, the term “guide RNA” relates to a synthetic fusion of two RNA molecules, a crRNA (CRISPR RNA) comprising a variable targeting domain, and a tracrRNA. In one embodiment, the guide RNA comprises a variable targeting domain of 12 to 30 nucleotide sequences and a RNA fragment that can interact with a Cas endonuclease.

As used herein, the term “guide polynucleotide”, relates to a polynucleotide sequence that can form a complex with a Cas endonuclease and enables the Cas endonuclease to recognize and optionally cleave a DNA target site (U.S. Provisional Application No. 62/023,239, filed Jul. 11, 2014). The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotride that solely comprises ribonucleic acids is also referred to as a “guide RNA”.

The guide polynucleotide can be a double molecule (also referred to as duplex guide polynucleotide) comprising a first nucleotide sequence domain (referred to as Variable Targeting domain or VT domain) that is complementary to a nucleotide sequence in a target DNA and a second nucleotide sequence domain (referred to as Cas endonuclease recognition domain or CER domain) that interacts with a Cas endonuclease polypeptide. The CER domain of the double molecule guide polynucleotide comprises two separate molecules that are hybridized along a region of complementarity. The two separate molecules can be RNA, DNA, and/or RNA-DNA-combination sequences. In some embodiments, the first molecule of the duplex guide polynucleotide comprising a VT domain linked to a CER domain is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the cRNA naturally occurring in Bacteria and Archaea. In one embodiment, the size of the fragment of the cRNA naturally occurring in Bacteria and Archaea that is present in a crNucleotide disclosed herein can range from, but is not limited to, 2, 3, 4, 5, 6, 7, 8, 9,10, 11, 12, 13, 14, 15, 16, 17, 18, 19, 20 or more nucleotides. In some embodiments the second molecule of the duplex guide polynucleotide comprising a CER domain is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides In one embodiment, the RNA that guides the RNA/Cas9 endonuclease complex, is a duplexed RNA comprising a duplex crRNA-tracrRNA.

The guide polynucleotide can also be a single molecule comprising a first nucleotide sequence domain (referred to as Variable Targeting domain or VT domain) that is complementary to a nucleotide sequence in a target DNA and a second nucleotide domain (referred to as Cas endonuclease recognition domain or CER domain) that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In some embodiments the single guide polynucleotide comprises a crNucleotide (comprising a VT domain linked to a CER domain) linked to a tracrNucleotide (comprising a CER domain), wherein the linkage is a nucleotide sequence comprising a RNA sequence, a DNA sequence, or a RNA-DNA combination sequence. The single guide polynucleotide being comprised of sequences from the crNucleotide and tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). In one embodiment of the disclosure, the single guide RNA comprises a CRNA or cRNA fragment and a tracrRNA or tracrRNA fragment of the type II/Cas system that can form a complex with a type II Cas endonuclease, wherein said guide RNA/Cas endonuclease complex can direct the Cas endonuclease to a plant genomic target site, enabling the Cas endonuclease to introduce a double strand break into the genomic target site. One aspect of using a single guide polynucleotide versus a duplex guide polynucleotide is that only one expression cassette needs to be made to express the single guide polynucleotide.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop seqence.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In certain embodiments the nucleotide sequence to be modified can be a regulatory sequence such as a promoter, wherein the editing of the promoter comprises replacing the promoter (also referred to as a “promoter swap” or “promoter replacement”) or promoter fragment with a different promoter (also referred to as replacement promoter) or promoter fragment (also referred to as replacement promoter fragment), wherein the promoter replacement results in any one of the following or any combination of the following: an increased promoter activity, an increased promoter tissue specificity, a decreased promoter activity, a decreased promoter tissue specificity, a new promoter activity, an inducible promoter activity, an extended window of gene expression, a modification of the timing or developmental progress of gene expression in the same cell layer or other cell layer (such as but not limiting to extending the timing of gene expression in the tapetum of maize anthers; see e.g. U.S. Pat. No. 5,837,850 issued Nov. 17, 1998), a mutation of DNA binding elements and/or deletion or addition of DNA binding elements. The promoter (or promoter fragment) to be modified can be a promoter (or promoter fragment) that is endogenous, artificial, pre-existing, or transgenic to the cell that is being edited. The replacement promoter (or replacement promoter fragment) can be a promoter (or promoter fragment) that is endogenous, artificial, pre-existing, or transgenic to the cell that is being edited.

Promoter elements to be inserted can be, but are not limited to, promoter core elements (such as, but not limited to, a CAAT box, a CCAAT box, a Pribnow box, a and/or TATA box, translational regulation sequences and/or a repressor system for inducible expression (such as TET operator repressor/operator/inducer elements, or SulphonylUrea (Su) repressor/operator/inducer elements. The dehydration-responsive element (DRE) was first identified as a cis-acting promoter element in the promoter of the drought-responsive gene rd29A, which contains a 9 bp conserved core sequence, TACCGACAT (Yamaguchi-Shinozaki, K., and Shinozaki, K. (1994) Plant Cell 6, 251 264). Insertion of DRE into an endogenous promoter may confer a drought inducible expression of the downstream gene. Another example is ABA-responsive elements (ABREs) which contain a (C/T) ACGTGGC consensus sequence found to be present in numerous ABA and/or stress-regulated genes (Busk P. K., Pages M. (1998) Plant Mol. Biol. 37:425-435). Insertion of 35S enhancer or MMV enhancer into an endogenous promoter region will increase gene expression (U.S. Pat. No. 5,196,525). The promoter (or promoter element) to be inserted can be a promoter (or promoter element) that is endogenous, artificial, pre-existing, or transgenic to the cell that is being edited.

In particular embodiments, wheat plants are used in the methods and compositions disclosed herein. As used herein, the term “wheat” refers to any species of the genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. mocha, T. compactum, T. sphaerococcum, T. vavilovii, and interspecies cross thereof. Tetraploid wheat includes T. durum (also referred to as durum wheat or Triticum turgidum ssp. durum), T. dicoccoides, T. dicoccum, T. polonicum, and interspecies cross thereof. In addition, the term “wheat” includes possible progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. A wheat cultivar for use in the present disclosure may belong to, but is not limited to, any of the above-listed species. Also encompassed are plants that are produced by conventional techniques using Triticum sp. as a parent in a sexual cross with a non-Triticum species, such as rye (Secale cereale), including but not limited to Triticale. In some embodiments, the wheat plant is suitable for commercial production of grain, such as commercial varieties of hexaploid wheat or durum wheat, having suitable agronomic characteristics which are known to those skilled in the art.

Typically, an intermediate host cell will be used in the practice of the methods and compositions disclosed herein to increase the copy number of the cloning vector. With an increased copy number, the vector containing the nucleic acid of interest can be isolated in significant quantities for introduction into the desired plant cells. In one embodiment, plant promoters that do not cause expression of the polypeptide in bacteria are employed.

Prokaryotes most frequently are represented by various strains of E. coli; however, other microbial strains may also be used. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang et al. (1977) Nature 198:1056), the tryptophan (trp) promoter system (Goeddel et al. (1980) Nucleic Acids Res. 8:4057) and the lambda derived P L promoter and N-gene ribosome binding site (Shimatake et al. (1981) Nature 292:128). The inclusion of selection markers in DNA vectors transfected in E coli. is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein disclosed herein are available using Bacillus sp. and Salmonella (Palva et al. (1983) Gene 22:229-235); Mosbach et al. (1983) Nature 302:543-545).

In some embodiments, the expression cassette or male-fertility polynucleotides disclosed herein are maintained in a hemizygous state in a plant. Hemizygosity is a genetic condition existing when there is only one copy of a gene (or set of genes) with no allelic counterpart. In certain embodiments, an expression cassette disclosed herein comprises a first promoter operably linked to a male-fertility polynucleotide which is stacked with a male-gamete-disruptive polynucleotide operably linked to a male-tissue-preferred promoter, and such expression cassette is introduced into a male-sterile plant in a hemizygous condition. When the male-fertility polynucleotide is expressed, the plant is able to successfully produce mature pollen grains because the male-fertility polynucleotide restores the plant to a fertile condition. Given the hemizygous condition of the expression cassette, only certain daughter cells will inherit the expression cassette in the process of pollen grain formation. The daughter cells that inherit the expression cassette containing the male-fertility polynucleotide will not develop into mature pollen grains due to the male-tissue-preferred expression of the stacked encoded male-gamete-disruptive gene product. Those pollen grains that do not inherit the expression cassette will continue to develop into mature pollen grains and be functional, but will not contain the male-fertility polynucleotide of the expression cassette and therefore will not transmit the male-fertility polynucleotide to progeny through pollen.

V. Modulating the Concentration and/or Activity of Male-Fertility Polypeptides

A method for modulating the concentration and/or activity of the male-fertility polypeptides disclosed herein in a plant is provided. The term “influences” or “modulates,” as used herein with reference to the concentration and/or activity of the male-fertility polypeptides, refers to any increase or decrease in the concentration and/or activity of the male-fertility polypeptides when compared to an appropriate control. In general, concentration and/or activity of a male-fertility polypeptide disclosed herein is increased or decreased by at least 1%, 5%, 10%, 20%, 30%, 40%, 50%, 60%, 70%, 80%, or 90% relative to a control plant, plant part, or cell. Modulation as disclosed herein may occur before, during and/or subsequent to growth of the plant to a particular stage of development. In specific embodiments, the male-fertility polypeptides disclosed herein are modulated in monocots, particularly wheat.

A variety of methods can be employed to assay for modulation in the concentration and/or activity of a male-fertility polypeptide. For instance, the expression level of the male-fertility polypeptide may be measured directly, for example, by assaying for the level of the male-fertility polypeptide or RNA in the plant (i.e., Western or Northern blot), or indirectly, for example, by assaying the male-fertility activity of the male-fertility polypeptide in the plant. Methods for measuring the male-fertility activity are described elsewhere herein or known in the art. In specific embodiments, modulation of male-fertility polypeptide concentration and/or activity comprises modulation (i.e., an increase or a decrease) in the level of male-fertility polypeptide in the plant. Methods to measure the level and/or activity of male-fertility polypeptides are known in the art and are discussed elsewhere herein. In still other embodiments, the level and/or activity of the male-fertility polypeptide is modulated in vegetative tissue, in reproductive tissue, or in both vegetative and reproductive tissue.

In one embodiment, the activity and/or concentration of the male-fertility polypeptide is increased by introducing the polypeptide or the corresponding male-fertility polynucleotide into the plant. Subsequently, a plant having the introduced male-fertility sequence is selected using methods known to those of skill in the art such as, but not limited to, Southern blot analysis, DNA sequencing, PCR analysis, or phenotypic analysis. In certain embodiments, marker polynucleotides are introduced with the male-fertility polynucleotide to aid in selection of a plant having or lacking the male-fertility polynucleotide disclosed herein. A plant or plant part altered or modified by the foregoing embodiments is grown under plant-forming conditions for a time sufficient to modulate the concentration and/or activity of the male-fertility polypeptide in the plant. Plant-forming conditions are well known in the art.

As discussed elsewhere herein, many methods are known in the art for providing a polypeptide to a plant including, but not limited to, direct introduction of the polypeptide into the plant, or introducing into the plant (transiently or stably) a polynucleotide construct encoding a male-fertility polypeptide. It is also recognized that the methods disclosed herein may employ a polynucleotide that is not capable of directing, in the transformed plant, the expression of a protein or an RNA. The level and/or activity of a male-fertility polypeptide may be increased, for example, by altering the gene encoding the male-fertility polypeptide or its promoter. Sec, e.g., Kmiec, U.S. Pat. No. 5,565,350; Zarling et al., PCT/US93/03868. Therefore mutagenized plants that carry mutations in male fertility genes, where the mutations modulate expression of the male fertility gene or modulate the activity of the encoded male-fertility polypeptide, are provided.

In certain embodiments, the concentration and/or activity of a male-fertility polypeptide is increased by introduction into a plant of an expression cassette comprising a male-fertility polynucleotide or an active fragment or variant thereof, as disclosed elsewhere herein. The male-fertility polynucleotide may be operably linked to a promoter that is heterologous to the plant or native to the plant. By increasing the concentration and/or activity of a male-fertility polypeptide in a plant, the male fertility of the plant is likewise increased. Thus, the male fertility of a plant can be increased by increasing the concentration and/or activity of a male-fertility polypeptide. For example, male fertility can be restored to a male-sterile plant by increasing the concentration and/or activity of a male-fertility polypeptide.

It is also recognized that the level and/or activity of the polypeptide may be modulated by employing a polynucleotide that is not capable of directing, in a transformed plant, the expression of a protein or an RNA. For example, the polynucleotides disclosed herein may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such nucleotide constructs and methods of use are known in the art. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Bectham et al. (1999) Proc. Natl. Acad. Sci. USA 96:8774-8778, herein incorporated by reference. In some embodiments, virus-induced gene silencing may be employed; see, for example, Ratcliff et al. (2001) Plant J. 25:237-245; Dinesh-Kumar et al. (2003) Methods Mol. Biol. 236:287-294; Lu et al. (2003) Methods 30:296-303; Burch-Smith et al. (2006) Plant Physiol. 142:21-27. It is therefore recognized that methods disclosed herein do not depend on the incorporation of the entire polynucleotide into the genome, only that the plant or cell thereof is altered as a result of the introduction of the polynucleotide into a cell.

In other embodiments, the level and/or activity of the polypeptide may be modulated by methods which do not require introduction of a polynucleotide into the plant, such as by exogenous application of dsRNA to a plant surface; see, for example, WO 2013/025670.

In one embodiment, the genome may be altered following the introduction of the polynucleotide into a cell. For example, the polynucleotide, or any part thereof, may incorporate into the genome of the plant. Alterations to the genome disclosed herein include, but are not limited to, additions, deletions, and substitutions of nucleotides into the genome. While the methods disclosed herein do not depend on additions, deletions, and substitutions of any particular number of nucleotides, it is recognized that such additions, deletions, or substitutions comprise at least one nucleotide.

### VI. Definitions

The term “wheat Ms26 gene” or similar reference means a gene or sequence in wheat that is orthologous to Ms26 in maize or rice, e.g. as disclosed in U.S. Pat. No. 7,919,676 or 8,293,970. Genomic DNA and polypeptide sequences of wheat Ms26 were disclosed in US patent publication 2014/0075597; the corresponding coding sequences are at SEQ ID Nos: 31-33 herein. Genomic DNA and polypeptide sequences of wheat Ms45 were disclosed in US patent publication 2014/0075597; the corresponding coding sequences are at SEQ ID Nos: 34-36 herein. Genomic DNA and polypeptide sequences of wheat Ms22 were disclosed in US patent publication 2014/0075597; the corresponding coding sequences are at SEQ ID Nos: 37-39 herein.

The term “allele” refers to one of two or more different nucleotide sequences that occur at a specific locus.

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

A “BAC”, or bacterial artificial chromosome, is a cloning vector derived from the naturally occurring F factor of Escherichia coli, which itself is a DNA element that can exist as a circular plasmid or can be integrated into the bacterial chromosome. BACs can accept large inserts of DNA sequence.

A “centimorgan” (“cM”) is a unit of measure of recombination frequency. One cM is equal to a 1% chance that a marker at one genetic locus will be separated from a marker at a second locus due to crossing over in a single generation.

A “chromosome” is a single piece of coiled DNA containing many genes that act and move as a unit during cell division and therefore can be said to be linked. It can also be referred to as a “linkage group”.

“Genetic markers” are nucleic acids that are polymorphic in a population and where the alleles of which can be detected and distinguished by one or more analytic methods, e.g., RFLP, AFLP, isozyme, SNP, SSR, HRM, and the like. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

“Genome” refers to the total DNA, or the entire set of genes, carried by a chromosome or chromosome set.

The term “genotype” is the genetic constitution of an individual (or group of individuals) defined by the allele(s) of one or more known loci that the individual has inherited from its parents. More generally, the term genotype can be used to refer to an individual's genetic make-up for all the genes in its genome.

A “locus” is a position on a chromosome, e.g. where a nucleotide, gene, sequence, or marker is located.

A “marker” is a means of finding a position on a genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker will consist of complementary primers flanking the locus and/or complementary probes that hybridize to polymorphic alleles at the locus. A DNA marker, or a genetic marker, can also be used to describe the gene, DNA sequence or nucleotide on the chromosome itself (rather than the components used to detect the gene or DNA sequence) and is often used when that DNA marker is associated with a particular trait in human genetics (e.g. a marker for breast cancer). The term marker locus refers to the locus (gene, sequence or nucleotide) that the marker detects.

Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected eg via DNA sequencing, PCR-based sequence specific amplification methods, detection of polynucleotide polymorphisms by allele specific hybridization (ASH), dynamic allele-specific hybridization (DASH), Competitive Allele-Specific Polymerase chain reaction (KASPar), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology have the advantage of being able to detect a series of linked SNP alleles that constitute a haplotype. Haplotypes tend to be more informative (detect a higher level of polymorphism) than SNPs.

A “marker allele”, alternatively an “allele detected by a marker” or “an allele at a marker locus”, can refer to one or a plurality of polymorphic nucleotide sequences found at a marker locus in a population.

A “marker locus” is a specific chromosome location in the genome of a species detected by a specific marker. A marker locus can be used to track the presence of a second linked locus, e.g., one that affects the expression of a phenotypic trait. For example, a marker locus can be used to monitor segregation of alleles at a genetically or physically linked locus, such as a QTL.

A “marker probe” is a nucleic acid sequence or molecule that can be used to identify the presence of an allele at a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus. Nucleic acids are “complementary” when they specifically “hybridize”, or pair, in solution, e.g., according to Watson-Crick base pairing rules.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A “polymorphism” is a variation in the DNA between 2 or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”.

A “reference sequence” or a “consensus sequence” is a defined sequence used as a basis for sequence comparison.

The articles “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this disclosure pertains, and all such publications and patent applications are herein incorporated by reference to the same extent as if each individual publication or patent application was specifically and individually indicated to be incorporated by reference.

### EXAMPLES

The following examples are offered to illustrate, but not to limit, the appended claims. It is understood that the examples and embodiments described herein are for illustrative purposes only and that persons skilled in the art will recognize various reagents or parameters that can be altered without departing from the spirit of the invention or the scope of the appended claims.

For these examples, wheat plants were grown and maintained under routine greenhouse conditions: seeds planted directly into soil, seedlings transferred to pots and exposed to 16 hours of daylight with temperatures ranging from 20-30° C.

Male fertility phenotyping used techniques known in the art. Screening for a male fertility phenotype in spring wheat was performed as follows: to prevent open-pollinated seeds from forming, 3 to 5 spikes were covered before anthesis with paper bags fastened with a paper clip and used for qualitative fertility scoring by visual inspection of developing microspores in anthers dissected from these spikes or by counting of seed resulting from self-fertilization.

Male fertility polynucleotides include the Ms26 polynucleotide and homologs and orthologs thereof. Ms26 polypeptides have been reported to have significant homology to P450 enzymes found in yeast, plants, and mammals. P450 enzymes have been widely studied and characteristic protein domains have been elucidated. The Ms26 protein contains several structural motifs characteristic of eukaryotic P450's, including a heme-binding domain, dioxygen-binding domain A, steroid-binding domain B, and domain C. Phylogenetic tree analysis revealed that Ms26 is most closely related to P450s involved in fatty acid omega-hydroxylation found in Arabidopsis thaliana and Vicia sativa. See, for example, US Patent Publication No. 2012/0005792, herein incorporated by reference. See also WO 2014/039815.

### Example 1

### Combining TaMS26 Mutations Results in Male Sterile Wheat

This example shows that combining mutations in the A, B and D genome of wheat Ms26 gene results in male sterile phenotype.

Single homozygous mutations in TaMs26-A, -B or -D

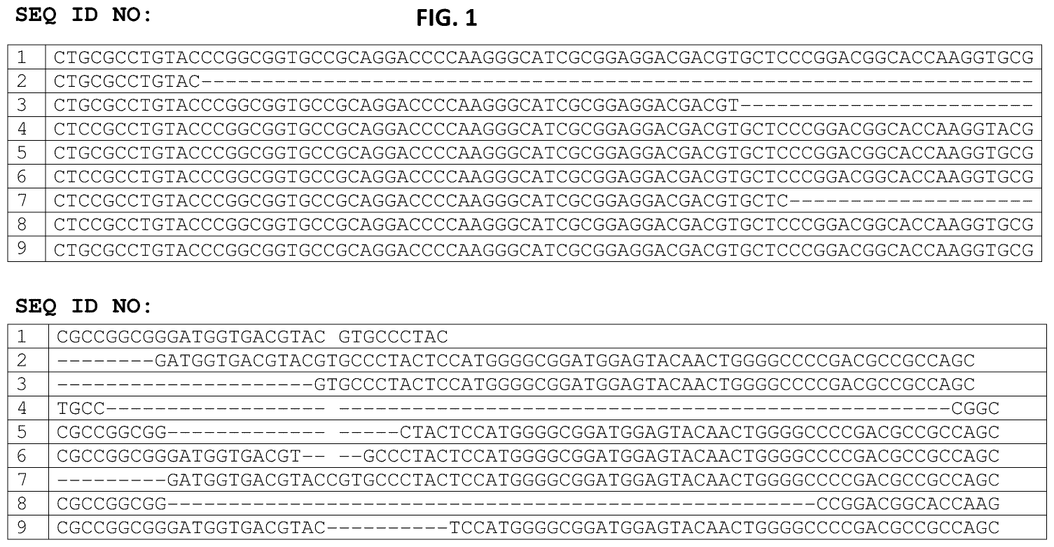

In the A, B or D genomic copy of the wheat Ms26 gene (WO2014/039815, FIG. 1 and Table 1), seven non-identical mutations have been generated and identified. The genetic nature of the Ms26 alleles present in hexaploid wheat plants is denoted as follows:
homozygous wild-type Ms26 alleles in genome A, B and D are represented by the designation Ms26^{A/B/D}.
homozygous deletion alleles are designated by a single number representing the deletion (or addition) present in the Ms26 genome copy; for example:
   the homozygous 4 bp deletion in the Ms26-A genome is represented as Ms26^{a4/B/D}.
   the homozygous 81 bp deletion present in the Ms26-B genome is represented as Ms26^{A/b81/D}.
heterozygous mutations are designated Ms26^{A:a4/B/D} and Ms26^{A/B:b81/D}, for example.

Plants which each contained one of the seven non-identical mutations shown in Table 1 were allowed to self-pollinate, to generate progeny plants that contained homozygous mutations upon which male fertility phenotypes were evaluated.

All plants containing a homozygous mutation in any one of the A, B or D genomic copy of the wheat Ms26 gene were completely male fertile and capable of generating selfed seed (Table 1). These results suggest that no single Ms26 genomic copy from the A, B or D genome is essential to confer function in wheat, as the other wild-type Ms26 copies still present in these plants function to maintain pollen development and a male fertile phenotype.

TABLE 1
Fertility phenotype associated with wheat plants
containing single-genome deletions in Ms26 alleles.

| Muta- | Seq ID | Sequence | | | Male |
|---|---|---|---|---|---|
| tion | No. | Change | GENOME | Ms26 allele | Fertility |
| 1 | 3 | GTAC Deletion | A | Ms26^{a4/B/D} | Fertile |
| 2 | 4 | C insert | A | Ms26^{a1/B/D} | Fertile |
| 3 | 5 | 9 bp Deletion | B | Ms26^{A/b9/D} | Fertile |
| 4 | 6 | 81 bp Deletion | B | Ms26^{A/b81/D} | Fertile |
| 5 | 7 | 23 bp Deletion | B | Ms26^{A/b23/D} | Fertile |
| 6 | 8 | 90 bp Deletion | D | Ms26^{A/B/d90} | Fertile |
| 7 | 9 | 96 bp Deletion | D | Ms26^{A/B/d96} | Fertile |

### Double Homozygous Mutations in TaMs26-A, -B or -D

To examine the impact on wheat male fertility when multiple TaMs26-A, -B or -D mutations are present in the same plant, mutations described in FIG. 1 were combined by crossing plants to generate different combinations of double homozygous mutant ms26 alleles. As shown in Table 2, double homozygous mutant pairs were generated which retained a single homozygous wild-type copy of TaMs26-A, -B or -D. All plants containing homozygous wild-type copies of only a single TaMs26-A, -B or -D allele generated pollen capable of self-fertilization. These plants produced seed numbers nearly identical to wild-type wheat Fielder controls (approximately 100-150 seed per plant). This result suggests that homozygous wild-type alleles derived from a single genome of TaMs26 are competent to maintain male fertility.

TABLE 2
Fertility phenotype associated with wheat plants
containing double genome deletions in Ms26 alleles.

| | | | | | Male |
|---|---|---|---|---|---|
| PLANT | Ms26-A | Ms26-B | Ms26-D | Ms26 | Fertility |
| 1 | GTAC | 81 bp | WT | Ms26^{a4/b81/D} | Fertile |
| | Deletion | Deletion | | | |
| 2 | GTAC | 23 bp | WT | Ms26^{a4/b23/D} | Fertile |
| | Deletion | Deletion | | | |
| 3 | WT | 9 bp | 96 bp | Ms26^{A/b9/d96} | Fertile |
| | | Deletion | Deletion | | |
| 4 | WT | 81 bp | 96 bp | Ms26^{A/b81/d96} | Fertile |
| | | Deletion | Deletion | | |
| 5 | GTAC | WT | 96 bp | Ms26^{a4/B/d96} | Fertile |
| | Deletion | | Deletion | | |

Moreover, plants that contained a TaMs26 homozygous deletion in one genome and a heterozygous wild-type allele in each of the other two genomes were also male fertile; for example, Ms26^{a4/B:b81/D:d90} plants contain homozygous 4-bp deletion alleles, wild-type and 81-bp deletion alleles, and wild-type and 90-bp deletion alleles in the TaMs26-A, B and D genome copies, respectively. These plants which combined homozygous deletions in a single genome with heterozygous wild-type alleles in the remaining two genomes were also male fertile and capable of producing nearly wild-type amounts of seed per plant (data not shown). This observation suggests that two wild-type Ms26 alleles, derived either from a single genome or from different genomes, are sufficient to support male fertility in wheat.

Triple homozygous mutations in TaMs26-A, -B and -D

Triple homozygous TaMs26-A, -B and -D mutant plants were also generated to examine the effect on wheat male fertility when none of the three genomes contained a functional copy of wheat Ms26. Plants containing triple TaMs26 heterozygous mutations were allowed to self-pollinate and progeny plants screened by PCR for either one of two genetic combinations of TaMs26: (1) a single genome Ms26 heterozygote plus a double (i.e. two-genome) homozygous ms26 mutant (Ms26^{A:a/b/d} or other combination) or (2) a triple homozygous ms26 mutant (ms26^{a/b/d}).

Spike heads from single genome heterozygous, double genome homozygous ms26 mutant plants, and from triple homozygous ms26 mutant plants, were covered before anthesis with paper bags and allowed to self-pollinate. Seed from these individual plants was pooled and counted as a qualitative measure of male fertility. As shown in Table 3. plants containing different combinations of triple homozygous ms26 mutations did not set self-seed. (Note, seed observed in two of these plants was likely to due to open fertilization as these heads were not bagged prior to anthesis.)

TABLE 3
Seed set associated with wheat plants containing
either triple genome deletions or single heterozygous,
double homozygous deletions in Ms26 alleles.

| Ms26-A | Ms26-B | Ms26-D | SEED SET-Fertility | | |
|---|---|---|---|---|---|
| 4 bp | 81 bp | 90 bp | | TOTAL | Seed per |
| Deletion | Deletion | Deletion | PLANTS | SEED | plant |
| HOM | HOM | HOM | 7 | 1* | 0.0 |
| HET | HOM | HOM | 19 | 122 | 6.4 |
| HOM | HET | HOM | 13 | 24 | 1.8 |
| HOM | HOM | HET | 9 | 164 | 18.2 |
| 4 bp | 23 bp | 90 bp | | | |
| Deletion | Deletion | Deletion | | | |
| HOM | HOM | HOM | 5 | 3* | 0.0 |
| HET | HOM | HOM | 11 | 56 | 5.1 |
| HOM | HET | HOM | 10 | 9 | 0.9 |
| HOM | HOM | HET | 1 | 27 | 27.0 |
| 4 bp | 81 bp | 96 bp | | | |
| Deletion | Deletion | Deletion | | | |
| HOM | HOM | HOM | 5 | 0 | 0 |
| HET | HOM | HOM | 7 | 32 | 4.6 |
| HOM | HET | HOM | 1 | 0 | 0.0 |
| HOM | HOM | HET | 9 | 105 | 11.7 |
| WT | WT | WT | 3 | 444 | 148.0 |

*spike head not covered to prevent open-pollination

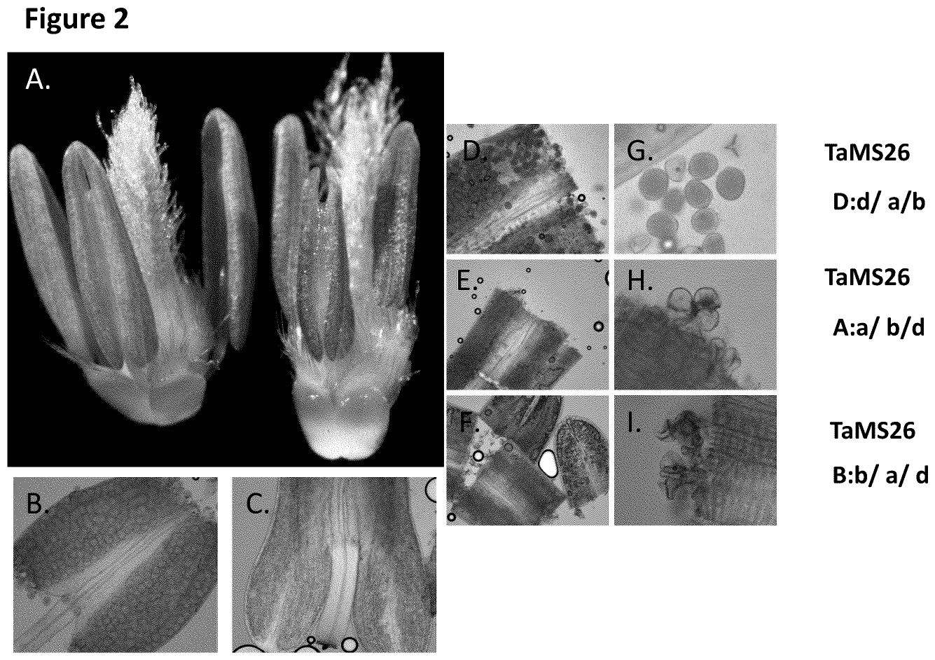

Flowers isolated from these triple homozygous ms26 plants are nearly identical to flowers from wild-type plants with the exception that anthers from the triple homozygous ms26 mutant (ms26^{a/b/d}) plants are visibly smaller in size when compared to anthers from wild-type plants (see FIG. 2A: wild-type flower on left side of panel, ms26^{a/b/d} flower on right side of panel).

Pollen development in these triple homozygous ms26 mutant plants was monitored by harvesting anthers at the late vacuolate stage of development. In other monocots, such as maize, rice and sorghum, mutations in the fertility gene Ms26 result in the breakdown of microspores shortly after quartet release (Loukides et al. (1995) Am. J. Bot. 82 (8): 1017-1023; Li et al. (2010) The Plant Cell Online 22 (1): 173-190.) As shown in FIG. 2B, anthers from wild-type wheat plants contain late vacuolate microspores, while microspores are absent in anthers from ms26^{a/b/d} plants (FIG. 2C).

It was also observed that microspore development varied and seed set was reduced in the single heterozygous, double homozygous ms26 mutant (Ms26^{A:a/b/d}) when compared either to wild-type Fielder plants (Ms26^{A/B/D})) or to plants homozygous for wild-type Ms26 alleles of a single genome (for example Ms26^{A/b/d}) or heterozygous at two genomes for wild-type and mutant Ms26 alleles (for example, but not limited to, Ms26^{A:a/B:b/d}). Microspore developmental differences (FIG. 2 D-F and G-I) were dependent upon the wild-type genomic Ms26 allele present and correlated well with observed differential seed set. For example, cross-sections of anthers derived from plants heterozygous for TaMs26-D (FIG. 2D), revealed developing microspores. Closer examination (FIG. 2G) identified morphological differences among the microspores contained in these anthers; while a proportion of these late vacuolate microspores appear rounded with well-defined walls, translucent, collapsed microspores are also easily detected. This is in contrast to the appearance of microspores from wild-type plants, where morphologically normal rounded vacuolate microspores are abundant and abnormal microspores are rare, if present at all. The presence of abnormally shaped microspores in heterozygous TaMs26-D anthers suggests that Ms26 function is likely reduced but not absent in these plants and the plant is competent to form morphologically normal appearing microspores. However, despite the presence of these developing microspores in heterozygous TaMs26-D anthers, seed set per plant (Table 3) was low (ranging from 12-27 seed per plant) when compared to plants containing wild-type TaMs26 alleles (100-150 seed per plant; see Table 3, WT) and suggests that a single wild-type allele of TaMs26 is not sufficient to fully restore male fertility. This observation is supported by examining microspore development in anthers derived from plants containing a single TaMs26-A or TaMs26-B allele. As shown in FIG. 2E and F, microspores are nearly absent in these anthers. In addition, only translucent, collapsed microspores are identified in anthers from wheat plants containing a single TaMs26-A allele (FIG. 2H), while only severely collapsed, translucent microspores are found in anthers from plants that contain a single wild-type allele from TaMs26-B (FIG. 2I). The observed impact on microspore viability was reflected in the low or no seed set from plants containing only a single TaMs26-A or TaMs26-B allele, respectively (Table 3).

Together these observations suggest that TaMs26 is an essential gene for wheat pollen development and, unexpectedly, the different genomic copies of TaMs26 are not equivalent in their ability to maintain male fertility when present as a single functional allele.

### Example 2

### A Single Copy of Monocot Ms26 Gene Cannot Restore Fertility of Triple Homozygous Mutations in TaMs26-A, -B and D Genome

To increase the ms26 male sterile inbred line, it would be advantageous to generate a maintainer line. To accomplish this, the maize Ms26 gene under control of the native maize Ms26 promoter (see, e.g., U.S. Pat. No. 7,098,388) was linked to maize alpha amylase under control of the maize PG47 promoter and to a DsRed2 gene under control of the barley LTP2 promoter (see, e.g., U.S. Pat. No. 5,525,716) and also carrying a PINII terminator sequence (Ms26-AA-DsRED). This construct was transformed directly into wheat by Agrobacterium-mediated transformation methods as referenced elsewhere herein, yielding several independent T-DNA insertion events for construct evaluation. Wheat plants containing single-copy ZmMs26-AA-DsRED cassette were emasculated, removing anthers, and stigmas fertilized with pollen from wheat plants heterozygous for the TaMS26-A, -B and -D alleles as described previously. Seeds were harvested, planted, and progeny screened by PCR to confirm hemizygous presence of ZmMs26-AA-DsRED and heterozygosity of TaMS26-A, -B and -D alleles and allowed to self-pollinate.

Red fluorescing seed from these selfed plants was planted, progeny screened by PCR to identify the genetic nature of the TaMS26-A, -B and -D alleles in these plants, the spike heads covered and allowed to self-pollinate. Seed from these individual plants was pooled and counted as a qualitative measure of male fertility. As shown in Table 4, in contrast to the low seed set observed in single genome heterozygous, double homozygous deletion plants (Ms26^{A:a/b/d} or other combination), increased seed set was observed when these plants contained a transformed copy of the ZmMs26-AA-DsRED cassette. This result demonstrates that the transformed copy of ZmMs26 associated with the two T-DNA insertions examined (E1 and E2), was functional, albeit at different efficiencies. Unexpectedly, however, in the absence of a functional endogenous TaMs26 allele (see triple homozygous ms26), neither ZmMs26-AA-DsRED T-DNA event examined restored full fertility, and no seeds were produced.

TABLE 4
Seed set in wheat plants containing a ZmMs26 complementation T-DNA insertion (E1 or E2)
and different combinations of ms26 genomic deletions. Nulls do not contain ZmMs26
complementation T-DNA insertion.

| | | | | SEED SET-Fertility | | |
|---|---|---|---|---|---|---|
| Ms26-A | Ms26-B | Ms26-D | Ms26 | | | Seed |
| 4 bp | 81 bp | 96 bp | complementation | | TOTAL | per |
| Deletion | Deletion | Deletion | event | PLANTS | SEED | plant |
| HOM | HOM | HOM | ZmMS26-E1 | 2 | 0 | 0.0 |
| HOM | HOM | HOM | ZmMS26-E2 | 3 | 0 | 0.0 |
| HET | HOM | HOM | ZmMS26-E1 | 3 | 330 | 110.0 |
| HET | HOM | HOM | ZmMS26-E2 | 5 | 173 | 34.6 |
| HOM | HET | HOM | ZmMS26-E1 | 3 | 399 | 133.0 |
| HOM | HET | HOM | ZmMS25-E2 | 2 | 110 | 55.0 |
| HOM | HOM | HET | ZmMS26-E1 | 4 | 482 | 120.5 |
| HOM | HOM | HET | ZmMS25-E2 | 1 | 58 | 58 |
| HET | HOM | HOM | Null | 7 | 32 | 4.6 |
| HOM | HET | HOM | Null | 1 | 0 | 0.0 |
| HOM | HOM | HET | Null | 9 | 105 | 11.7 |

### Approaches to Restore Male Fertility in Wheat Plants Containing Triple Homozygous Mutations in TaMs26-A, -B and -D Using a Transformed Copy or Copies of an Ms26 Gene

The inability of the transformed ZmMs26 to restore male fertility when present in single copy was an unexpected result. In this example, strategies are described to overcome the inability of a wild-type Ms26 gene to restore fertility to wheat plants containing triple homozygous mutations in Ms26.

Based on the observation that a single genomic copy of the wheat Ms26 was only partially sufficient to restore male fertility when other genomic Ms26 alleles are mutant, and that plants are male fertile when a transformed copy of an Ms26 gene is combined with this single endogenous wild-type allele, increasing expression or activity of the transformed copy of the Ms26 gene may restore male fertility in ms26 triple homozygous mutant plants. Increasing expression could be accomplished in several ways. For example, the promoter used to express the ZmMs26 gene, or any other Ms26 gene, could be replaced or modified such that the duration or level, or both, of the transcribed Ms26 gene would increase. Transcriptional enhancer elements could also be used to achieve increased Ms26 expression. Other changes could include modifications of the structural gene which result in improved splicing of the primary transcript, improved translational efficiency of the encoded mRNA such as by removal of mRNA destabilizing elements, optimizing translation initiation or elongation, or the addition or removal of sequences to result in an increased half-life of the primary encoded RNA or the spliced transcript. Different sources of Ms26 genes could be used, for example from, but not limited to, wheat, rice, barley, sorghum, Brachypodium, Arabidopsis, Setaria;or the ZmMs26 structural gene could be altered to result in a protein with increased P450 enzymatic activity; or some or all of the above described changes could be combined.

Another strategy that could be employed would be to increase the copy number of Ms26 present in the transformation cassette so that multiple Ms26 genes, when present in ms26 plants, would result in Ms26-encoded P450 function at levels sufficient to restore male fertility. The multiple copies could include, but are not limited to, similar genes or Ms26 genes from different species. In addition, modifications described above, such as promoter replacement or modification, or enhancement of transcription, translation or mRNA processing or stability, could also be incorporated singly or duplexed into the multiple Ms26 copies described in this copy-number strategy.

Yet another strategy that could be employed to confer sufficient Ms26 transformation-cassette-encoded P450 function competent to restore male fertility would be to use genomic alleles of wheat Ms26 that are reduced, but not abolished, in function. The mutations described in the above examples are loss-of-function alleles with fertility restoration dependent upon which single wild-type allele remains. For example, plants containing only a wild-type TaMs26-B allele are male sterile when paired with the two deletion alleles of TaMs26-A and -D; however fertility was restored with the addition of the transformed Ms26 copy in this genetic background. This result suggests that the TaMs26-B allele is functional but not to a level sufficient to restore fertility. In contrast to deletion mutations in alleles of TaMs26 which render Ms26 non-functional, gene mutations which reduce Ms26 expression or encoded P450 protein activity could be used in strategies to overcome the inability of a transformed Ms26 gene to restore male fertility. In this strategy, sequence changes in the endogenous TaMs26 gene(s) would result in low levels of Ms26-encoded P450 expression or activity, incapable of conferring male fertility unless combined with a transformed copy of Ms26. Sequence differences in one, two or all three endogenous TaMs26 alleles could be isolated or generated and combined such that, only in the presence of a transformed copy of Ms26, male fertility is restored. These mutations in the endogenous Ms26 gene could result in the reduction of transcribed mRNA as a result of alterations to promoter, splice site, mRNA stabilization, or mRNA termination sequences. In addition, single or multiple changes could be made within the Ms26 gene to result in a newly encoded P450 polypeptide with reduced activity, to reduce but not abolish Ms26 function, and could be used as an alternative to loss-of-function alleles described previously.

### Increasing Capacity for Restoration of Male Fertility in Wheat Plants Containing Triple Homozygous Mutations in TaMs26-A, -B and -D

The previous observation that male fertility can be restored when a transformed copy of an Ms26 gene is combined with a single endogenous wild-type allele suggested that increasing expression of the transformed copy of the Ms26 gene may restore male fertility in ms26 triple homozygous mutant plants. Increasing expression could be accomplished in any of several ways. In this example the maize 5126 anther-specific promoter was used to express the ZmMs26 gene, to increase the duration or level, or both, of the transcribed Ms26 gene.

To accomplish this, the maize Ms26 gene under control of the native maize 5126 promoter (see, e.g., U.S. Pat. No. 5,689,051) was linked to maize alpha amylase gene under control of the maize PG47 promoter and to a DsRed2 gene under control of the barley LTP2 promoter (sec, e.g., U.S. Pat. No. 5,525,716) and also carrying a PINII terminator sequence (Zm5126:Ms26-AA-DsRED). This construct was transformed directly into wheat genotypes homozygous for TaMS26-B and -D mutations but wild type for TaMS26-A (Ms26^{A/b/d}) by Agrobacterium-mediated transformation methods as referenced elsewhere herein, yielding several independent T-DNA insertion events for construct evaluation. Of these T0 Ms26^{A/b/d} plants, those containing a single-copy Zm5126:ZmMs26-AA-DsRED cassette were emasculated, removing anthers, and stigmas fertilized with pollen from wheat plants heterozygous for the TaMS26-A, -B and -D alleles as described previously. Seeds were harvested, planted, and T1 progeny screened by PCR to confirm hemizygous presence of ZmMs26-AA-DsRED and zygosity of TaMS26-A, -B and -D alleles and allowed to self-pollinate. Red fluorescing seed from these selfed plants was planted, T2 progeny screened by PCR to identify the genetic nature of the TaMS26-A, -B and -D alleles in these plants, the spike heads covered and allowed to self-pollinate. Seed was counted as a qualitative measure of male fertility. As shown in Table 5, three events (E1, E2, E3) produced fertile plants. This demonstrates that the Zm5126:Ms26-AA-DsRED construct is functional as it can complement the single-heterozygous/double-homozygous genotype. Failure of event E4 to restore fertility and partial restoration of fertility in event E3 may be due to reduced or impaired expression of the Zm5126: Ms26-AA-DsRED construct, for example due to transgene integrity issue or location of the transgene insertion.

TABLE 5
Seed set in wheat plants comprising a Zm5126:
ZmMs26 complementation T-DNA insertion

| Ms26-A | Ms26-B | Ms26-D | Ms26 | | |
|---|---|---|---|---|---|
| 4 bp | 81 bp | 96 bp | complementation | | Seed Set- |
| Deletion | Deletion | Deletion | event | PLANTS | Fertility |
| HET | HOM | HOM | Zm5126: | 2 | Fertile |
| | | | ZmMS26- | | |
| | | | E1 (T1) | | |
| HET | HOM | HOM | Zm5126: | 2 | Fertile |
| | | | ZmMS26- | | |
| | | | E2 (T1) | | |
| HET | HOM | HOM | Zm5126: | 14 | 4 Fertile/ |
| | | | ZmMS26- | | 10 Sterile |
| | | | E3 (T1) | | |
| HOM | HOM | HOM | Zm5126: | 2 | Sterile |
| | | | ZmMS26- | | |
| | | | E4 (T2) | | |
| HET | HOM | HOM | Zm5126: | 7 | Sterile |
| | | | ZmMS26- | | |
| | | | E4 (T2) | | |
| HOM | HOM | HET | Zm5126: | 10 | Sterile |
| | | | ZmMS26- | | |
| | | | E4 (T2) | | |
| HET | HOM | HOM | Null | 1 | Sterile |
| HOM | HOM | HOM | Null | 1 | Sterile |
| HOM | HOM | HET | Null | 1 | Sterile |

### Example 3

Generation of Nutations in TaMs26-A, -B and -D Homeologs using CRISPR-CAS System

To obtain additional mutations in TaMs26-A, -B and -D genes, a monocot-codon-optimized Cas9 gene from Streptococcus pyogenes M1 GAS (SF370) (Patent Application US 2015/0082478 A1) was used. The potato ST-LS1 intron was introduced in order to eliminate expression in E. coli and Agrobacterium. To facilitate nuclear localization of the Cas9 protein in plant cells, Simian virus 40 (SV40) monopartite amino terminal nuclear localization signal (MAPKKKRKV; SEQ ID NO: 10) and Agrobacterium tumefaciens bipartite VirD2 T-DNA border endonuclease carboxyl terminal nuclear localization signal (KRPRDRHDGELGGRKRAR; SEQ ID NO: 11) were incorporated at the amino and carboxyl-termini of the Cas9 open reading frame respectively. The monocot-optimized Cas9 gene was operably linked to a maize constitutive promoter by standard molecular biological techniques. To confer efficient guide RNA expression (or expression of the duplexed crRNA and tracrRNA) in wheat, the maize U6 polymerase III promoter and maize U6 polymerase III terminator were operably fused to the termini of a guide RNA using standard molecular biology techniques.

A 21 nucleotide crRNA molecule (gacgtacgtgccctactccat; SEQ ID NO: 12) containing a region complementary to one strand of the double strand DNA target (referred to as the variable targeting domain) was designed upstream of a PAM sequence for target site recognition and cleavage (Gasiunas et al. (2012) Proc. Natl. Acad. Sci. USA 109: E2579-86, Jinek et al. (2012) Science 337:816-21, Mali et al. (2013) Science 339:823-26, and Cong et al. (2013) Science 339:819-23). Guide RNA (gRNA) also consisted of a 77 nucleotide tracrRNA fusion transcript used to direct Cas9 to cleave sequence of interest. The construct also included a DsRed2 gene under control of the maize Ubiquitin promoter (see, e.g., U.S. Pat. No. 5,525,716) and PINII terminator for selection during transformation. This construct was transformed directly into wheat by Agrobacterium-mediated transformation methods as referenced elsewhere herein, yielding several independent T-DNA insertion events for construct evaluation. T0 wheat plants containing one-or two-copy transgene are grown to maturity and seed harvested. T1 plants are grown and examined for the presence of NHEJ mutations by deep sequencing.

In other embodiments, other DNA sequences which are recognized by S. pyogenes Cas9 protein are used to direct mutagenesis of wheat Ms26, reducing or abolishing gene function and thereby impacting male fertility.

### Example 4

### Targeted Mutations at Gene Encoding Cytochrome P450 Family Protein, MS26, in Rice Using Cas9/gRNA System

Cas9/guideRNA (Cas9/gRNA) mediated targeted genome modification is demonstrated in rice by knocking out ms26 gene. The gRNAs were designed by selecting the target sequences in different regions of exon 2. The guides designed were cloned into either rice (Os) scaffold or maize (Zm) U6 scaffold as indicated in Table 6. Two sets of experiments were conducted: 1) to check the efficiency of different gRNAs by co-bombarding with Cas9 protein construct in rice callus tissue and 2) to check the efficiency of selected gRNA in stable transgenic rice plants. Callus events co-bombarded with different gRNAs and Cas9 protein were analysed for indels in the targeted region. Similarly, plants harbouring stable rice events generated using selected gRNA sequence (ACGTACGTGCCCTACTCCAT; SEQ ID NO: 13) were also analysed for indels at ms26 locus. Based on the illumina® data obtained, indels (SDN1) at rice ms26 locus have been observed in both callus events and stable lines. Using the Os-U3 PolIII promoter, 35 out of 45 callus events analyzed were mutated at ms26 locus (78%). With Zm-U6 PolIII promoter, 17 out of 19 callus events analyzed were mutated at ms26 locus (98%). In stable transgenic lines, 19 events out of 35 analyzed were mutated (55.9%). In both the experiments, mono-allelic as well as bi-allelic mutations have been observed; the bi-allelic mutations are predominant (Tables 7 and 8). The majority of the mutations observed were short indels (<20 bps) with relatively higher percentage of single bp deletion (Table 9).

Phenotyping of rice events indicated that there is no fertile pollen formation in ms26 mutant lines. There was no seed recovered from selfed plants, but seeds were recovered from mutant lines after crossing with WT pollen donor. The data obtained clearly indicated that the Cas9/gRNA system efficiently created mutations at ms26 locus, which resulted in male sterility.

TABLE 6
gRNA sequences used in co-bombardment experiments.

| | | | SEQ |
|---|---|---|---|
| Gene | | | ID |
| Name | Locus ID | Guide sequences | NO: |
| MS26 | LOC_ | ACGTACGTGCCCTACTCCAT (OsU3) | 13 |
| | Os03g07250 | ACGTACGTGCCCTACTCCA (OsU3) | 14 |
| | | ATCGAGCTCGGGGAGGCCGG (OsU3) | 15 |
| | | ATGAAGAGCCCCATGG (OsU3) | 16 |
| | | GACGTACGTGCCCTACTCCAT (ZmU6) | 17 |
| | | GACGTACGTGCCCTACTCCA (ZmU6) | 18 |

TABLE 7
ms26 mutation data obtained from rice calli
co-bombarded with Cas9 and gRNA constructs.

| Mutation rate with Os-U3 | | | | Mutation rate with Zm-U6 | | | |
|---|---|---|---|---|---|---|---|
| | Mutant | | | | Mutant | | |
| Events | events | Mono- | Bi- | Events | events | Mono- | Bi- |
| screened | (%) | allelic | allelic | Screened | (%) | allelic | allelic |
| 45 | 35 (78%) | 13 (37%) | 22 (63%) | 19 | 17 (89%) | 8 (47%) | 9 (60%) |

TABLE 8
Mutation data obtained from rice stable events transformed
with Cas9/gRNA construct targeted to MS26 gene (gRNA
sequence: ACGTACGTGCCCTACTCCAT (SEQ ID NO: 13)).

| Events | Mutant events | Mono-allelic | Bi-allelic |
|---|---|---|---|
| screened | (%) | (%) | (%) |
| 34 | 19 (55.9) | 8 (42.1) | 11 (57.9) |

TABLE 9
Frequency of different types of mutations (indels)
obtained at ms26 locus using Cas9/gRNA system.

| Indel type | | Percent of total |
|---|---|---|
| 1 | bp | 62 |
| 2 | bp | 7 |
| 3 | bp | 5 |
| 6-10 | bp | 12 |
| >10 | bp | 14 |

1. A method of controlling male fertility in a polyploid species, comprising modulating expression of a male fertility gene differentially across genomes.

2. The method of claim 1, wherein the species is wheat.

3. The method of claim 2, wherein the gene is Ms26.

4. The method of claim 3, wherein two genomes are homozygous for the recessive allele of Ms26 and the third genome is heterozygous for the dominant allele of Ms26.

5. The method of claim 4, wherein expression is modulated by transforming the plant with a transgenic construct comprising an Ms26 polynucleotide encoding an Ms26 polypeptide.

6. The method of claim 3, wherein two genomes are homozygous for the recessive allele of Ms26 and the third genome is homozygous for the dominant allele of Ms26.

7. The method of claim 6, wherein expression is modulated by transforming the plant with a transgenic construct comprising an Ms26 polynucleotide encoding a functional Ms26 polypeptide.

8. The method of claim 3, wherein all three genomes are homozygous for the recessive allele of Ms26.

9. The method of claim 8, wherein expression is modulated by transforming the plant with a transgenic construct comprising an Ms26 polynucleotide encoding a functional Ms26 polypeptide.

10. A male-sterile wheat plant comprising double or triple homozygous mutations in a gene encoding a gene product necessary for male fertility.

11. The plant of claim 10, further comprising a transgenic construct comprising a polynucleotide encoding a polypeptide which restores male fertility to the plant.

12. The plant of claim 10, wherein the gene is Ms26.

13. The plant of claim 11, wherein the transgenic construct comprises an Ms26 polynucleotide.

14. The plant of claim 13, wherein the Ms26 polynucleotide is native to a species other than wheat.

15. The plant of claim 11, wherein the transgenic construct further comprises
(a) A promoter operably linked to the polynucleotide encoding a polypeptide which restores male fertility to the plant, wherein said promoter drives expression in the plant;
(b) A pollen-specific promoter operably linked to a polynucleotide encoding a gene product which interferes with starch accumulation; and
(c) A seed-specific promoter operably linked to a polynucleotide encoding a marker protein.

16. The plant of claim 4, wherein expression of the dominant allele of Ms26 is enhanced by one or more of the methods selected from the group consisting of: modification of the promoter; operable linkage to a different promoter; incorporation of transcriptional enhancer elements in the construct; modification of the structural gene to improve splicing of the primary transcript; removal of mRNA destabilizing elements, optimization of translation initiation or elongation; and addition or removal of sequences to increase the half-life of the primary encoded RNA or the spliced transcript.

17. The plant of claim 11, wherein expression of the polynucleotide is enhanced by one or more of the methods selected from the group consisting of: modification of the promoter;
operable linkage to a different promoter; incorporation of transcriptional enhancer elements in the construct; modification of the structural gene to improve splicing of the primary transcript; removal of mRNA destabilizing elements, optimization of translation initiation or elongation; and addition or removal of sequences to increase the half-life of the primary encoded RNA or the spliced transcript.

18. A method for modifying expression of Ms26 in a wheat plant by modifying a target site in a wheat Ms26 gene, the method comprising providing a guide crRNA molecule to a plant cell having a Cas endonuclease, wherein said guide RNA and Cas endonuclease are capable of forming a complex that enables the Cas endonuclease to introduce a double strand break at said target site in the Ms26 gene.

19. The method of claim 18, wherein said guide crRNA molecule has the sequence of SEQ ID NO: 12.
